(19) United States
(12) Patent Application Publication
(10) Pub. No.: US 2012/0028981 A1
(43) Pub. Date: Feb. 2, 2012

(54) Kinase Knockdown Via Electrophilically Enhanced Inhibitors

(75) Inventor: Richard Miller, Portola Valley, CA (US)

(73) Assignee: PRINCIPIA BIOPHARMA INC., Menlo Park, CA (US)

(21) Appl. No.: 13/127,705

(22) PCT Filed: Nov. 5, 2009
(86) PCT No.: PCT/US2009/063431

§ 371 Date: Jun. 17, 2011

Related U.S. Application Data
(63) Continuation-in-part of application No. 12/265,594, filed on Nov. 5, 2008, now abandoned.

(51) Int. Cl. A61K 31/5377; C07D 277/56; C07D 417/14; C07D 413/12; C07D 263/48; C07D 401/12; C07D 233/90; A61K 31/427; A61K 31/426; A61K 31/506; A61K 31/454; A61K 31/422; A61K 31/421; A61K 31/4168; A61K 31/496; C12N 9/99; A61P 35/00; A61P 29/00; C07D 417/12

(52) U.S. Cl. 514/236.8; 548/194; 544/327; 546/209; 548/234; 544/133; 546/210; 548/332.1; 514/370; 514/256; 514/326; 514/377; 514/398; 514/253.1; 435/184

(57) ABSTRACT

Provided herein are electrophilically enhanced kinase inhibitors. Also provided herein are methods of making and utilizing the same.

### BACKGROUND OF THE INVENTION

Kinases play critical roles in signaling pathways controlling fundamental cellular processes such as proliferation, differentiation, and death (apoptosis).

### CROSS-REFERENCE

This application claims the benefit of U.S. application Ser. No. 12/265,594, filed Nov. 5, 2008, which application is incorporated herein by reference in its entirety.

### SUMMARY OF THE INVENTION

Described herein are kinase inhibitors that bind irreversibly to kinases that contain a nucleophilic amino acid residue near the ATP-binding site of the kinase. Such kinase inhibitors include an electrophilic moiety that reacts with the nucleophilic amino acid residue to form a covalent bond. Further such kinase inhibitors include a moiety that binds non-covalently to the ATP-binding site of the kinase. In other words, such kinase inhibitors include a non-covalent ATP-binding site moiety and an electrophilic moiety that react with a nucleophilic amino acid residue to form a covalent bond. In certain embodiments, the nucleophilic amino acid residue is a cysteine residue. In certain embodiments, the effect of such kinase inhibitors is to knockdown such kinases so that such a kinase is no longer reactive with at least one native substrate or ligand.

In some embodiments, such kinase inhibitors reversibly bind to kinases that do not contain a nucleophilic amino acid residue near the ATP-binding site of such a kinase, but irreversibly bind to kinases that do have a nucleophilic amino acid residue near the ATP-binding site.

Also described herein are the use of such kinase inhibitors for the treatment of diseases or conditions in which the activity of a kinase having a nucleophilic amino acid reside near its ATP-binding site contributes to the etiology or the symptoms of such a disease or condition. Administration of such kinase inhibitors irreversibly inhibits (or knockdown) the activity of such a kinase and provide therapeutic benefit to an individual afflicted with such a disease or condition.

Also described herein are kinase inhibitors that irreversibly inhibit kinases that have a nucleophilic amino acid residue near its ATP-binding site and which are reversibly inhibited by N-(2-chloro-6-methylphenyl)-2-[[4-(2-hydroxyethyl)-1-piperazinyl]-2-methyl-4-pyrimidinyl]amino]-5-thiazole carboxamide (“Compound 100”). In certain embodiments, are kinase inhibitors that reversibly inhibit kinases that do not have a nucleophilic amino acid residue near its ATP-binding site and which are reversibly inhibited by Compound 100.

In certain of any of the aforementioned embodiments, the nucleophilic amino acid residue is a cysteine and the kinase is a tyrosine kinase.

Provided in certain embodiments here in are compounds of Formula I:

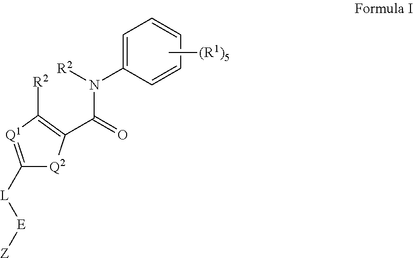

wherein:
each R¹ is independently H, alkyl, halo, hydroxy, alkoxy, cyano, nitro, C(═X)YR², or YC(═X)R²;
   each X is independently S or O;
   each Y is independently S or O;
each R² is independently H or alkyl;
L is Aₙ, wherein
   each A is independently NR¹, S(O)ₘ, O, C(═X)Y, Y(C═X), substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl; wherein each m is independently 0-2;
   n is 0-5;
Q¹ is N or CR²;
Q² is NR², S, or O;
E is an electrophile;
Z is —(Z¹)ₚ—Z² or is absent,
   Z¹ is NR³, O, C(═X)Y, Y(C═X), substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl
   Z² is H, NR³₂, S(O)ₘR³, OR³, —C(═X)YR³, —Y(C═X)R³, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
   each R³ is independently H, halo, hydroxy, alkoxy, cyano, nitro, —C(═X)YR⁴, —YC(═X)R⁴, substituted or unsubstituted alkyl, substituted or unsubstituted aryl, substituted or unsubstituted cycloalkyl, substituted or unsubstituted heteroaryl, substituted or unsubstituted heterocycloalkyl, or substituted or unsubstituted heteroalkyl, wherein R⁴ is substituted or unsubstituted alkyl, substituted or unsubstituted aryl, substituted or unsubstituted cycloalkyl, substituted or unsubstituted heteroaryl, substituted or unsubstituted heterocycloalkyl, or substituted or unsubstituted heteroalkyl;
   p is 0-4;
or a pharmaceutically acceptable salt thereof.

In certain embodiments, E is an electrophile subject to nucleophilic substitution or nucleophilic addition when contacted with a thiol and/or a thiolate.

In specific embodiments, provided herein are compounds of Formula I,
wherein E is:
—(CR¹¹R¹²)ᵣ—(CR⁵═CR⁵)_{q}—(CR¹¹R¹²)ᵣ—
   wherein
      R¹¹ and R¹² are independently H, CN, NO₂, substituted or unsubstituted alkyl, substituted or unsubstituted aryl, or substituted or unsubstituted heteroaryl, or taken together are ═S, ═N—OR⁸, or ═O; wherein each R⁸ is independently substituted or unsubstituted alkyl, substituted or unsubstituted aryl, substituted or unsubstituted cycloalkyl, substituted or unsubstituted heteroaryl, substituted or unsubstituted heterocycloalkyl, or substituted or unsubstituted heteroalkyl;
      each R⁵ is independently H, halo, hydroxy, alkoxy, cyano, nitro, S(O)₁₋₂R⁸, —C(═X)YR⁸, —YC(═X)R⁸, substituted or unsubstituted alkyl, substituted or unsubstituted aryl, substituted or unsubstituted cycloalkyl, substituted or unsubstituted heteroaryl, substituted or unsubstituted heterocycloalkyl, substituted or unsubstituted heteroalkyl, or two R⁵ are taken together to form a bond;
      each r is independently 0-2;
      q is 0-2;
—(CR⁶R⁷)—X²
   wherein
      R⁶ and R⁷ are independently H, halo, hydroxy, alkoxy, cyano, nitro, —C(═X)YR⁸, —YC(═X)R⁸, substituted or unsubstituted alkyl, substituted or unsubstituted aryl, substituted or unsubstituted cycloalkyl, substituted or unsubstituted heteroaryl, substituted or unsubstituted heterocycloalkyl, substituted or unsubstituted heteroalkyl, or is a bond to Z; or R⁶ and R⁷ taken together are ═O or ═S;
      X² is halo, OR⁹, NR⁹ᵥ, N₃, SR⁹, or SCN; wherein R⁹ is —(S(O)ₜ)ᵤ—R¹⁰; wherein each R¹⁰ is independently H, substituted or unsubstituted alkyl, substituted or unsubstituted aryl, substituted or unsubstituted cycloalkyl, substituted or unsubstituted heteroaryl, substituted or unsubstituted heterocycloalkyl, or substituted or unsubstituted heteroalkyl; or X² and R⁷ when taken together with the carbon to which they are bound form an oxirane or oxetane; wherein t is 1-2, wherein u is 0-1, wherein v is 2-3;
—NR⁸(C═O)O—; —O(C═O)NR⁸—; —CR⁸R¹³(C═O)—; or —CR⁸R¹³(C═O)—, wherein R¹³ is halo.

In certain specific embodiments, each R¹ is independently H, halo or alkyl. In some embodiments, Q¹ is N. In certain embodiments, n is 1-2. In some embodiments, E is —(C═O)—(CH═CH)—, —(CH═CH)—(C═O)—, —C(CN)═CH—, —CH═C(CN)—, —C(NO₂)═CH—, or —CH═C(NO₂)—. In certain embodiments, n is 2, and wherein one A is tetrahydroquinolinyl, quinolinyl, tetrahydroisoquinolinyl, isoquinolinyl, pyridinyl, piperizinyl, or morpholino. In some embodiments, Z² is a substituted or unsubstituted piperizinyl, or a substituted or unsubstituted morpholino.

In some specific embodiments, provided herein are compounds of Formula I having the Formula II:

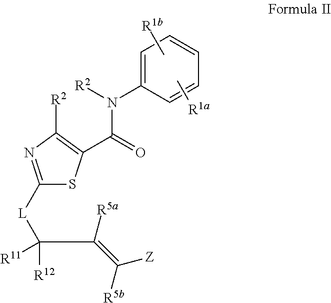

wherein
R^{1a} is H, halo, or lower alkyl;
R^{2a} is H, halo, or lower alkyl;
R¹¹ is H;
R¹² is H; or R¹¹ and R¹² taken together are ═O;
R^{5a} is H, lower alkyl, CN, NO₂, or SO₂R⁸; and
R^{5b} is H, CN, NO₂, or SO₂R⁸.

In specific embodiments, R^{1a} is CH₃ and R^{1b} is Cl. In further or alternative embodiments, R¹¹ and R¹² taken together are ═O, and R^{5a} and R^{5b} are H.

Also provided in certain embodiments herein is a pharmaceutical composition comprising a therapeutically effective amount of a compound of Formula I

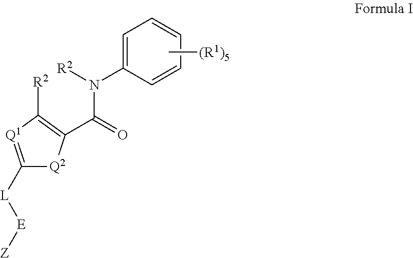

wherein:
each R¹ is independently H, alkyl, halo, hydroxy, alkoxy, cyano, nitro, C(═X)YR², or YC(═X)R²;
each X is independently S or O;
each Y is independently S or O;
each R² is independently H or alkyl;
L is Aₙ, wherein
   each A is independently NR¹, S(O)ₘ, O, C(═X)Y, Y(C═X), substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl; wherein each m is independently 0-2;
   n is 0-5;
Q¹ is N or CR²;
Q² is NR², S, or O;
E is an electrophile;
Z is —(Z¹)ₚ—Z² or is absent,
   Z¹ is NR³, O, C(═X)Y, Y(C═X), substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl
   Z² is H, NR³₂, S(O)ₘR³, OR³, —C(═X)YR³, —Y(C═X)R³, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
   each R³ is independently H, halo, hydroxy, alkoxy, cyano, nitro, —C(═X)YR⁴, —YC(═X)R⁴, substituted or unsubstituted alkyl, substituted or unsubstituted aryl, substituted or unsubstituted cycloalkyl, substituted or unsubstituted heteroaryl, substituted or unsubstituted heterocycloalkyl, or substituted or unsubstituted heteroalkyl, wherein R⁴ is substituted or unsubstituted alkyl, substituted or unsubstituted aryl, substituted or unsubstituted cycloalkyl, substituted or unsubstituted heteroaryl, substituted or unsubstituted heterocycloalkyl, or substituted or unsubstituted heteroalkyl;
   p is 0-4;
or a pharmaceutically acceptable salt thereof;
   and a pharmaceutically acceptable carrier.

Provided in some embodiments herein is a method of treating a disorder mediated by a cysteine containing kinase comprising administering to an individual in need thereof a therapeutically effective amount of a compound of Formula I:

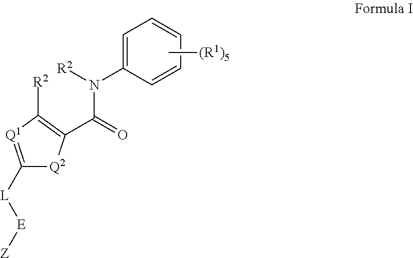

wherein:
each R¹ is independently H, alkyl, halo, hydroxy, alkoxy, cyano, nitro, C(═X)YR², or YC(═X)R²;
each X is independently S or O;
each Y is independently S or O;
each R² is independently H or alkyl;
L is Aₙ, wherein
   each A is independently NR¹, S(O)ₘ, O, C(═X)Y, Y(C═X), substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl; wherein each m is independently 0-2;
   n is 0-5;
Q¹ is N or CR²;
Q² is NR², S, or O;
E is an electrophile;
Z is —(Z¹)ₚ—Z² or is absent,
   Z¹ is NR³, O, C(═X)Y, Y(C═X), substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl
   Z² is H, NR³₂, S(O)ₘR³, OR³, —C(═X)YR³, —Y(C═X)R³, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
   each R³ is independently H, halo, hydroxy, alkoxy, cyano, nitro, —C(═X)YR⁴, —YC(═X)R⁴, substituted or unsubstituted alkyl, substituted or unsubstituted aryl, substituted or unsubstituted cycloalkyl, substituted or unsubstituted heteroaryl, substituted or unsubstituted heterocycloalkyl, or substituted or unsubstituted heteroalkyl, wherein R⁴ is substituted or unsubstituted alkyl, substituted or unsubstituted aryl, substituted or unsubstituted cycloalkyl, substituted or unsubstituted heteroaryl, substituted or unsubstituted heterocycloalkyl, or substituted or unsubstituted heteroalkyl;
   p is 0-4;
or a pharmaceutically acceptable salt thereof.

In specific embodiments, the cysteine containing kinase comprises a cysteine in proximity to the ATP binding site of the kinase. In some embodiments, the cysteine containing kinase is BTK, BMX, TEC, TXK, ITK, EGFR, ErbB2, ErbB4, JAK3, or BLK. In certain embodiments, the disorder is cancer, an inflammatory disorder, or an autoimmune disorder mediated by the cysteine containing kinase.

Provided in some embodiments herein is a method of binding a cysteine containing kinase to a compound of Formula I comprising contacting the kinase with the compound of Formula I, wherein the compound of Formula I has the structure:

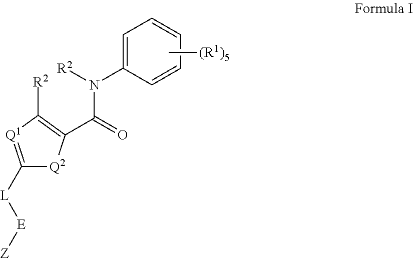

wherein:
each R¹ is independently H, alkyl, halo, hydroxy, alkoxy, cyano, nitro, C(═X)YR², or YC(═X)R²;
each X is independently S or O;
each Y is independently S or O;
each R² is independently H or alkyl;
L is Aₙ, wherein
   each A is independently NR¹, S(O)ₘ, O, C(═X)Y, Y(C═X), substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl; wherein each m is independently 0-2;
   n is 0-5;
Q¹ is N or CR²;
Q² is NR², S, or O;
E is an electrophile;
Z is —(Z¹)ₚ—Z² or is absent,
   Z¹ is NR³, O, C(═X)Y, Y(C═X), substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl
   Z² is H, NR³₂, S(O)ₘR³, OR³, —C(═X)YR³, —Y(C═X)R³, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
   each R³ is independently H, halo, hydroxy, alkoxy, cyano, nitro, —C(═X)YR⁴, —YC(═X)R⁴, substituted or unsubstituted alkyl, substituted or unsubstituted aryl, substituted or unsubstituted cycloalkyl, substituted or unsubstituted heteroaryl, substituted or unsubstituted heterocycloalkyl, or substituted or unsubstituted heteroalkyl, wherein R⁴ is substituted or unsubstituted alkyl, substituted or unsubstituted aryl, substituted or unsubstituted cycloalkyl, substituted or unsubstituted heteroaryl, substituted or unsubstituted heterocycloalkyl, or substituted or unsubstituted heteroalkyl;
   p is 0-4;
or a pharmaceutically acceptable salt thereof.

In specific embodiments, the cysteine containing kinase is BTK, BMX, TEC, TXK, ITK, EGFR, ErbB2, ErbB4, JAK3, or BLK. In further or alternative embodiments, the kinase is contacted with the compound of Formula I in vivo.

### DETAILED DESCRIPTION OF THE FIGURES

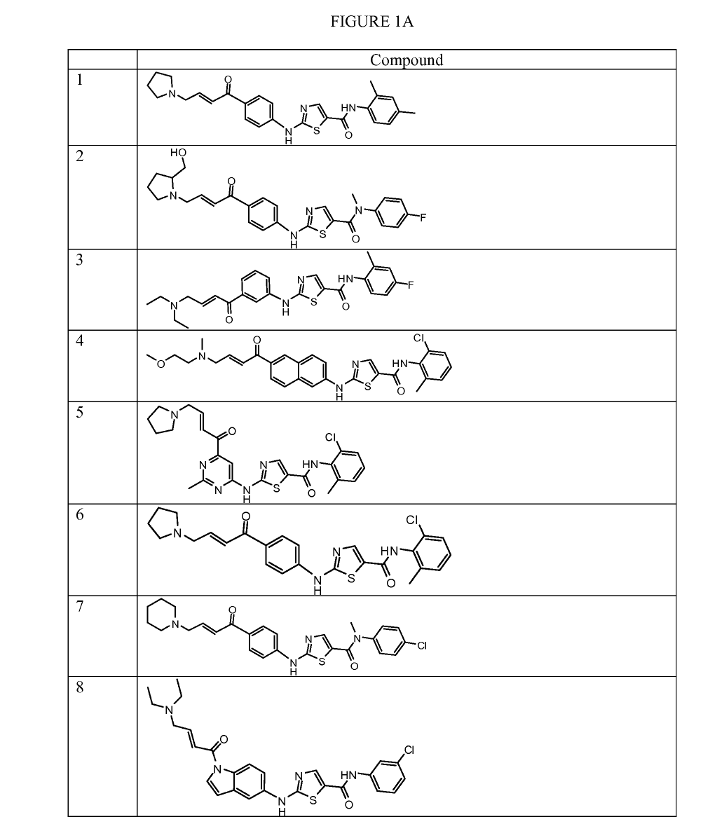

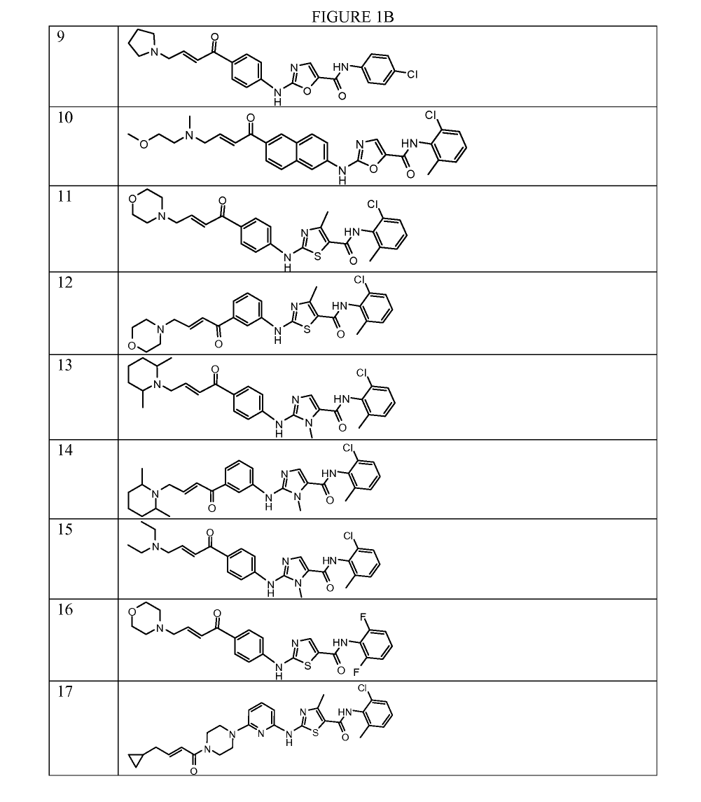

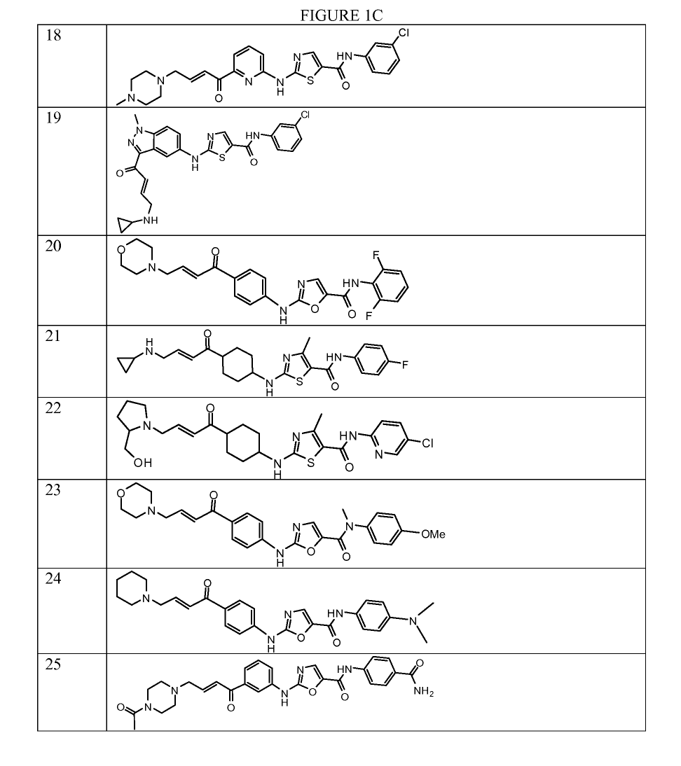

FIG. 1 presents illustrative examples of compounds described herein.

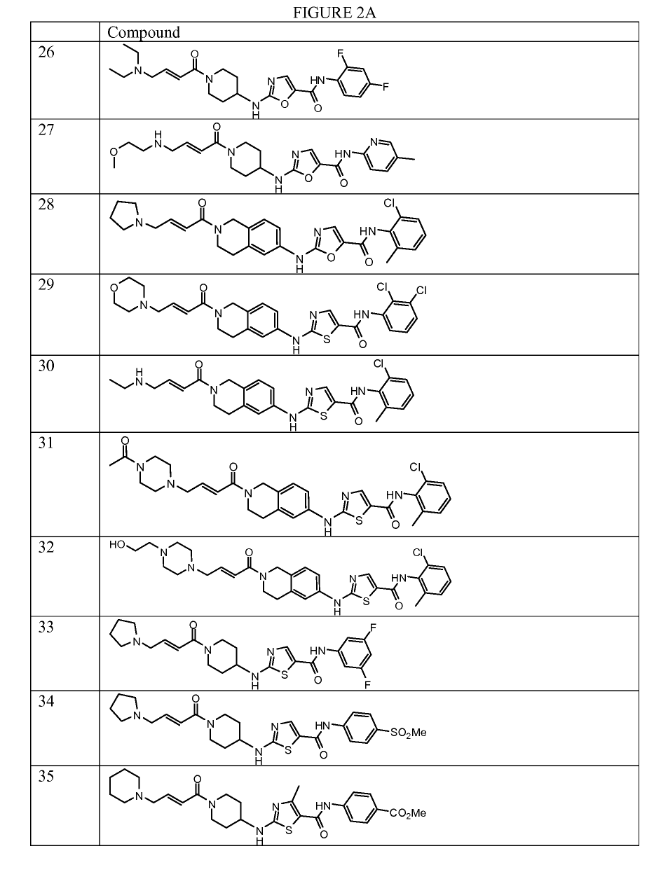

FIG. 2 presents illustrative examples of compounds described herein.

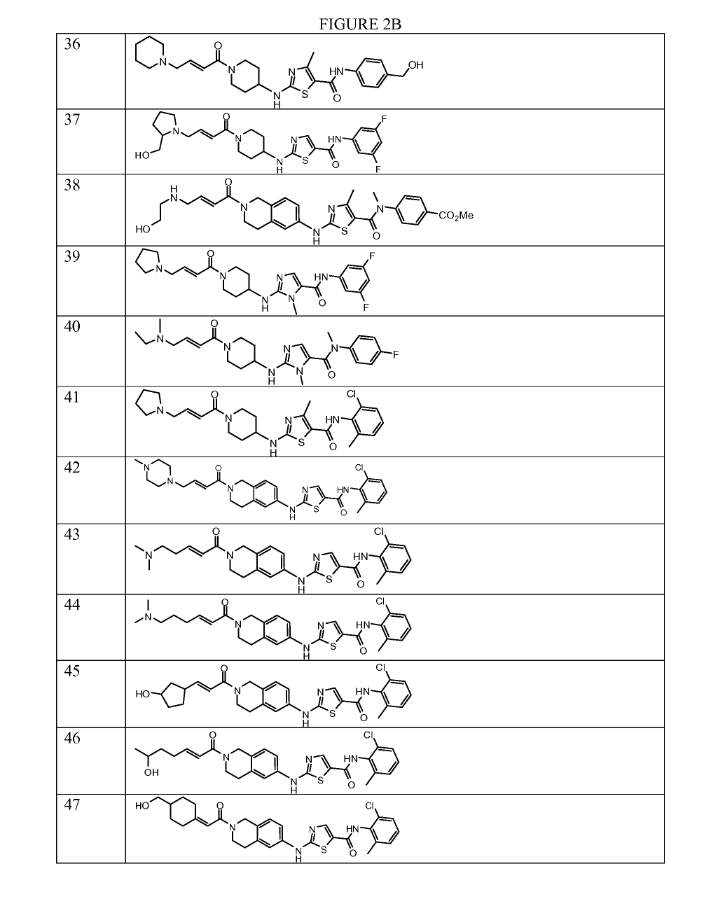

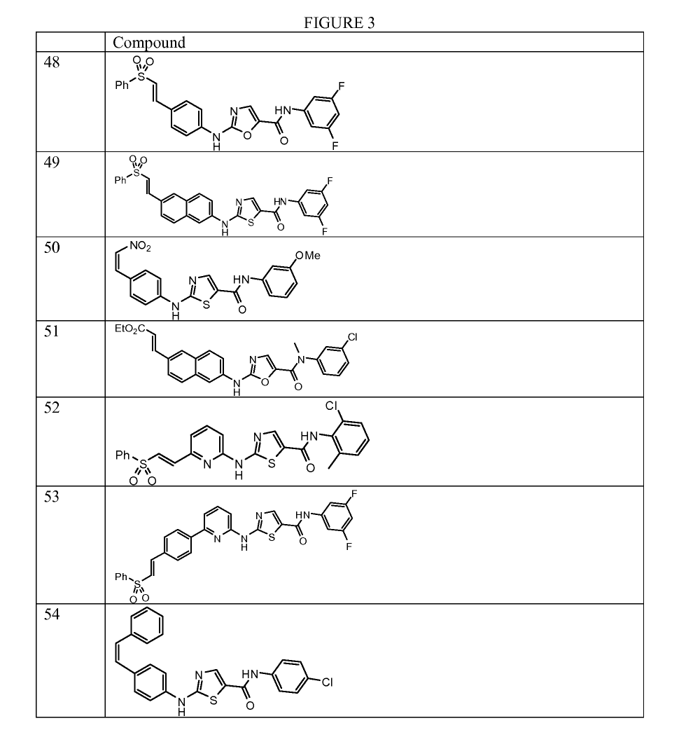

FIG. 3 presents illustrative examples of compounds described herein.

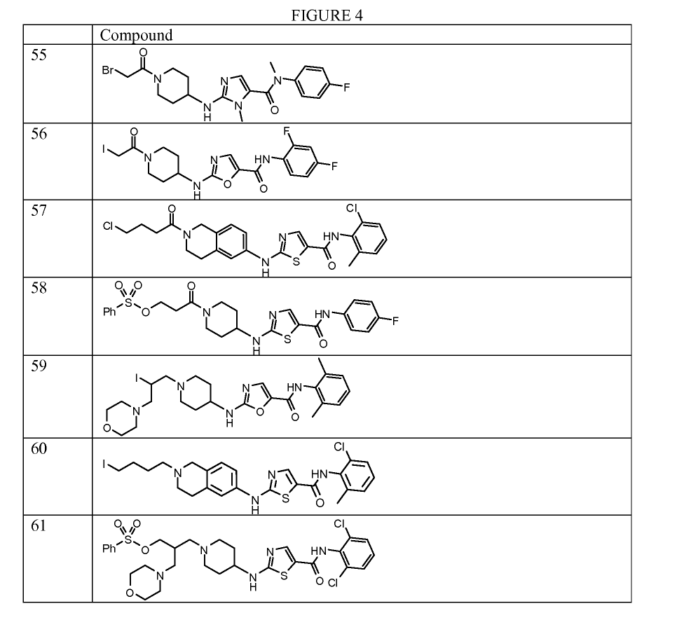

FIG. 4 presents illustrative examples of compounds described herein.

### DETAILED DESCRIPTION OF THE INVENTION

Provided herein are electrophilically enhanced kinase inhibitors. In some embodiments, provided herein are compounds related to Compound 100. In some embodiments, the compounds provided herein are electrophilically enhanced analogs of Compound 100. Certain compounds provided herein are Compound 100 analogues modified or substituted to comprise an electrophilic group. In specific embodiments, the Compound 100 analogues are modified or substituted with the electrophilic group at a site that does not affect the ability of the compound to bind the ATP binding site of a kinase (e.g., tyrosine kinase). In certain embodiments, the electrophilic group is a group that undergoes nucleophilic substitution or nucleophilic addition when in proximity to a thiol, a thiolate, a cysteine residue, or any one or more of such groups. In some embodiments, provided herein are compounds that are irreversible inhibitors of a cysteine containing kinases (e.g., cysteine containing kinases with a cysteine spatially near an ATP-binding site of the kinase). Moreover, provided herein are compounds that are reversible inhibitors of kinases that do not comprise a cysteine spatially near an ATP-binding site of the kinase.

In specific embodiments, provided herein is a compound of Formula I:

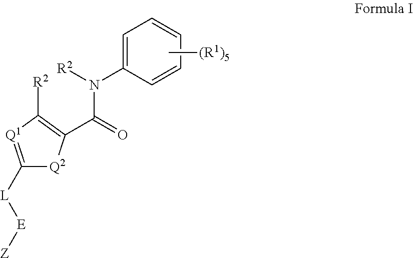

wherein:
each R¹ is independently H, substituted or unsubstituted alkyl, substituted or unsubstituted heteroalkyl, halo, hydroxy, substituted or unsubstituted alkoxy, substituted or unsubstituted amino, substituted or unsubstituted aryl, substituted or unsubstituted heteroaryl, substituted or unsubstituted heterocycloalkyl, cyano, nitro, C(═X)YR², or YC(═X)R²;
   each X is independently NR², S or O;
   each Y is independently NR², S or O;
each R² is independently H, halo, hydroxy, substituted or unsubstituted alkyl, substituted or unsubstituted aryl, substituted or unsubstituted heterocycloalkyl, substituted or unsubstituted heteroaryl, substituted or unsubstituted cycloalkyl, substituted or unsubstituted heteroalkyl;
L is Aₙ, wherein
   each A is independently NR¹, S(O)ₘ, O, C(═X)Y, Y(C═X), substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl; wherein each m is independently 0-2;
   n is 0-5;
Q¹ is N or CR²;
Q² is NR², S, or O;
E is an electrophile;
Z is —(Z¹)ₚ—Z² or is absent,
   Z¹ is NR³, O, C(═X)Y, Y(C═X), substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
   Z² is H, NR³₂, S(O)ₘR³, OR³, —C(═X)YR³, —Y(C═X)R³, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
   each R³ is independently H, halo, hydroxy, alkoxy, cyano, nitro, —C(═X)YR⁴, —YC(═X)R⁴, substituted or unsubstituted alkyl, substituted or unsubstituted aryl, substituted or unsubstituted cycloalkyl, substituted or unsubstituted heteroaryl, substituted or unsubstituted heterocycloalkyl, or substituted or unsubstituted heteroalkyl, wherein R⁴ is substituted or unsubstituted alkyl, substituted or unsubstituted aryl, substituted or unsubstituted cycloalkyl, substituted or unsubstituted heteroaryl, substituted or unsubstituted heterocycloalkyl, or substituted or unsubstituted heteroalkyl;
   p is 0-4;
or a pharmaceutically acceptable salt thereof.

In some embodiments, the electrophile is or comprises a group that is subject to nucleophilic substitution or nucleophilic addition when contacted with a thiol, a thiolate, a cysteine residue or two or more of the same. In certain embodiments, the electrophile is a Michael accepting group. In some embodiments, the electrophile is a group comprising a carbon attached to a leaving group. Any suitable leaving group is used herein, including, by way of non-limiting example, OR⁹, NR⁹ᵥ, N₃, SR⁹, or SCN; wherein R⁹ is —(S(O)ₜ)ᵤ—R¹⁰; wherein each R¹⁰ is independently H, substituted or unsubstituted alkyl, substituted or unsubstituted aryl, substituted or unsubstituted cycloalkyl, substituted or unsubstituted heteroaryl, substituted or unsubstituted heterocycloalkyl, or substituted or unsubstituted heteroalkyl, wherein t is 1-2, wherein u is 0-1, and wherein v is 2-3. In certain embodiments, the electrophilic group is a group comprising an oxirane or oxetane.

In specific embodiments, E is or comprises —(CR¹¹R)ᵣ—(CR⁵═CR⁵)_{q}—(CR¹¹R¹²)ᵣ—. In some embodiments, R¹¹ and R¹² are independently H, CN, NO₂, substituted or unsubstituted alkyl, substituted or unsubstituted aryl, or substituted or unsubstituted heteroaryl, or taken together are ═S, ═N—OR⁸, or ═O. In certain embodiments, each R⁸ is independently substituted or unsubstituted alkyl, substituted or unsubstituted aryl, substituted or unsubstituted cycloalkyl, substituted or unsubstituted heteroaryl, substituted or unsubstituted heterocycloalkyl, or substituted or unsubstituted heteroalkyl. In some embodiments, each R⁵ is independently H, halo, hydroxy, alkoxy, cyano, nitro, S(O)₁₋₂R⁸, —C(═X)YR⁸, —YC(═X)R⁸, substituted or unsubstituted alkyl, substituted or unsubstituted aryl, substituted or unsubstituted cycloalkyl, substituted or unsubstituted heteroaryl, substituted or unsubstituted heterocycloalkyl, substituted or unsubstituted heteroalkyl, or two R⁵ are taken together to form a bond. In certain embodiments, each r is independently 0-2. In some embodiments, each q is independently 0-2.

In some embodiments, E is or comprises —(CR⁶R⁷)—X². In certain embodiments, each R⁶ and R⁷ are independently H, halo, hydroxy, alkoxy, cyano, nitro, —C(═X)YR⁸, —YC(═X)R⁸, substituted or unsubstituted alkyl, substituted or unsubstituted aryl, substituted or unsubstituted cycloalkyl, substituted or unsubstituted heteroaryl, substituted or unsubstituted heterocycloalkyl, substituted or unsubstituted heteroalkyl, or is a bond to Z. In certain embodiments, R⁶ and R⁷ taken together are ═O or ═S. In some embodiments, X² is any suitable leaving group. In certain embodiments, X² is a leaving group that is subject to nucleophilic substitution with a thiol, thiolate, cysteine residue, or any one or more of the same. In specific embodiments, X² is a halo, OR⁹, NR⁹ᵥ, N₃, SR⁹, or SCN. In some embodiments, each R⁹ is independently —(S(O)ₜ)ᵤ—R¹⁰. In certain embodiments, each R¹⁹ is independently H, substituted or unsubstituted alkyl, substituted or unsubstituted aryl, substituted or unsubstituted cycloalkyl, substituted or unsubstituted heteroaryl, substituted or unsubstituted heterocycloalkyl, or substituted or unsubstituted heteroalkyl. In some embodiments, X² and R⁷ when taken together with the carbon to which they are bound form an oxirane or oxetane. In some embodiments, t is 1-2. In certain embodiments, u is 0-1. In some embodiments, v is 2-3.

In certain embodiments, E is or comprises —NR⁸(C═O)O—. In other embodiments, E is —O(C═O)NR⁸—. In still other embodiments, is or comprises —CR⁸R¹³(C═O)—. In yet other embodiments, E is or comprises —CR⁸R¹³(C═O)—. In certain embodiments, R¹³ is a leaving group, e.g., a halo.

In some embodiments, each R¹ is independently H, halo or alkyl. In certain embodiments, Q¹ is N. In some embodiments, Q¹ is CR², e.g., CH. In some embodiments, Q² is S or O. In specific embodiments, Q¹ is N and Q² is NR². In other specific embodiments, Q¹ is N and Q² is O. In still other specific embodiments, Q¹ is CR² and Q² is O. In yet other specific embodiments, Q¹ is CR² and Q² is S. In still other specific embodiments, Q¹ is N and Q² is S.

In some embodiments, n is 1-2. In certain embodiments, n is 0, 1, 2, 3, 4, or 5. In some embodiments, p is 0, 1, 2, 3, or 4.

In specific embodiments, E is —(C═O)—(CH═CH)—, —(CH═CH)—(C═O)—, —C(CN)═CH—, —CH═C(CN)—, —C(NO₂)═CH—, or —CH═C(NO₂)—. In specific embodiments, one A is NR¹, and another A is substituted or unsubstituted heterocyclo. In more specific embodiments, one A is substituted or unsubstituted tetrahydroquinolinyl, substituted or unsubstituted quinolinyl, substituted or unsubstituted tetrahydroisoquinolinyl, substituted or unsubstituted isoquinolinyl, substituted or unsubstituted pyridinyl, substituted or unsubstituted piperizinyl, or substituted or unsubstituted morpholino. In more specific embodiments, L is a NR¹-substituted or unsubstituted heterocyclo. In certain embodiments, L has and/or is selected to have a size and/or length sufficient to provide the electrophile (E) in proximity to a cysteine residue when a compound described herein is in an ATP binding pocket of a kinase (e.g., BTK, BMX, TEC, TXK, ITK, EGFR, ErbB2, ErbB4, JAK3, and/or BLK). In some embodiments, Z² is a substituted or unsubstituted piperizinyl, or a substituted or unsubstituted morpholino. In certain embodiments, Z is a bulky group that reduces the electrophilic reactivity of E. In more specific embodiments, Z is a group (e.g., group with slight to moderate sterics) that increases the specificity of a compound for a cysteine group of a kinase to which it is bound. In still more specific embodiments, Z reduces reactivity with other cysteine groups (e.g., serum cysteine groups), while not significantly altering the reactivity of the electrophile (E) for the cysteine group of the kinase to which it is bound.

In specific embodiments, each R¹ is independently H, alkyl, halo, hydroxy, alkoxy, cyano, nitro, C(═X)YR², or YC(═X)R². In some embodiments, each R² is independently H or alkyl.

In specific embodiments provided herein are compounds of any of FIGS. 1-4.

In some embodiments, provided herein is a compound of the Formula II:

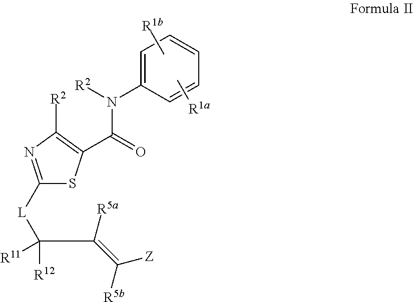

wherein
R^{1a} is H, halo, or lower alkyl;
R^{2a} is H, halo, or lower alkyl;
R¹¹ is H or lower alkyl;
R¹² is H or lower alkyl;
   or R¹¹ and R¹² taken together are ═O, ═S, or NR²;
R^{5a} is H, lower alkyl, CN, NO₂, or SO₂R⁸; and
R^{5b} is H, lower alkyl, CN, NO₂, or SO₂R⁸.

In certain embodiments, R², Z and L are as defined above. In specific embodiments, L is a NR¹-substituted or unsubstituted heterocycloalkyl, NR¹-substituted or unsubstituted heteroaryl, NR¹-substituted or unsubstituted aryl, or

NR¹-substituted or unsubstituted cycloalkyl. In specific embodiments, Z is substituted or unsubstituted alkyl-heterocycloalkyl, substituted or unsubstituted heteroalkyl, substituted or unsubstituted alkyl, or substituted or unsubstituted alkyl-cycloalkyl.

In some embodiments, R^{1a} is lower alkyl, e.g., CH₃. In certain embodiments, R^{1b} is halo, e.g., Cl. In certain embodiments, R^{1a} is CH₃, and R^{1b} is Cl. In some embodiments, R^{1a} is CH₃, and R^{1b} is CH₃. In certain embodiments, R^{1a} is Cl, and R^{1b} is Cl. In some embodiments, R^{5b} is H, CN, NO₂, or SO₂R⁸. In some embodiments, R¹¹ and R¹² taken together are ═O. In certain embodiments, R^{5a} and R^{5b} are H.

In certain embodiments, provided herein is a compound of the Formula III:

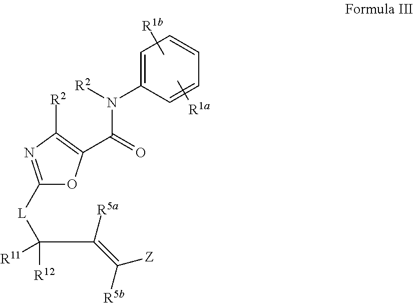

wherein
R^{1a} is H, halo, or lower alkyl;
R^{2a} is H, halo, or lower alkyl;
R¹¹ is H or lower alkyl;
R¹² is H or lower alkyl;
   or R¹¹ and R¹² taken together are ═O, ═S, or NR²;
R^{5a} is H, lower alkyl, CN, NO₂, or SO₂R⁸; and
R^{5b} is H, lower alkyl, CN, NO₂, or SO₂R⁸.

In certain embodiments, R², Z and L are as defined above. In specific embodiments, L is a NR¹-substituted or unsubstituted heterocycloalkyl, NR¹-substituted or unsubstituted heteroaryl, NR¹-substituted or unsubstituted aryl, or

NR¹-substituted or unsubstituted cycloalkyl. In specific embodiments, Z is substituted or unsubstituted alkyl-heterocycloalkyl, substituted or unsubstituted heteroalkyl, substituted or unsubstituted alkyl, or substituted or unsubstituted alkyl-cycloalkyl.

In some embodiments, R^{1a} is lower alkyl, e.g., CH₃. In certain embodiments, R^{1b} is halo, e.g., Cl. In certain embodiments, R^{1a} is CH₃, and R^{1b} is Cl. In some embodiments, R^{1a} is CH₃, and R^{1b} is CH₃. In certain embodiments, R^{1a} is Cl, and R^{1b} is Cl. In some embodiments, R^{5b} is H, CN, NO₂, or SO₂R⁸. In some embodiments, R¹¹ and R¹² taken together are ═O. In certain embodiments, R^{5a} and R^{5b} are H.

In certain embodiments, provided herein is a compound of the Formula IV:

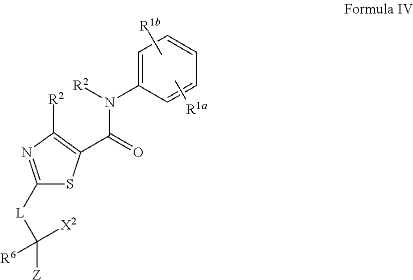

wherein
R^{1a} is H, halo, or lower alkyl;
R^{2a} is H, halo, or lower alkyl;
R⁶ is H, lower alkyl, or halo; and
X² is a halo, OR⁹, NR⁹ᵥ, N₃, SR⁹, or SCN.

In certain embodiments, R², R⁹, v, Z and L are as defined above. In specific embodiments, L is a NR¹-substituted or unsubstituted heterocycloalkyl, NR¹-substituted or unsubstituted heteroaryl, NR¹-substituted or unsubstituted aryl, or

NR¹-substituted or unsubstituted cycloalkyl. In specific embodiments, Z is substituted or unsubstituted alkyl-heterocycloalkyl, substituted or unsubstituted heteroalkyl, substituted or unsubstituted alkyl, or substituted or unsubstituted alkyl-cycloalkyl.

In some embodiments, provided herein is a compound of the Formula V:

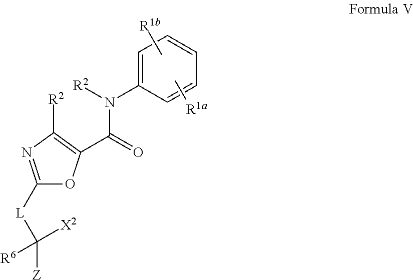

wherein
R^{1a} is H, halo, or lower alkyl;
R^{2a} is H, halo, or lower alkyl;
R⁶ is H, lower alkyl, or halo; and
X² is a halo, OR⁹, NR⁹ᵥ, N₃, SR⁹, or SCN.

In certain embodiments, R², R⁹, v, Z and L are as defined above. In specific embodiments, L is a NR¹-substituted or unsubstituted heterocycloalkyl, NR¹-substituted or unsubstituted heteroaryl, NR¹-substituted or unsubstituted aryl, or

NR¹-substituted or unsubstituted cycloalkyl. In specific embodiments, Z is substituted or unsubstituted alkyl-heterocycloalkyl, substituted or unsubstituted heteroalkyl, substituted or unsubstituted alkyl, or substituted or unsubstituted alkyl-cycloalkyl.

In certain embodiments, compounds described herein have one or more chiral centers. As such, all stereoisomers are envisioned herein. In various embodiments, compounds described herein are present in optically active or racemic forms. It is to be understood that the compounds of the present invention encompasses racemic, optically-active, regioisomeric and stereoisomeric forms, or combinations thereof that possess the therapeutically useful properties described herein. Preparation of optically active forms is achieve in any suitable manner, including by way of non-limiting example, by resolution of the racemic form by recrystallization techniques, by synthesis from optically-active starting materials, by chiral synthesis, or by chromatographic separation using a chiral stationary phase. In some embodiments, mixtures of one or more isomer is utilized as the therapeutic compound described herein. In certain embodiments, compounds described herein contains one or more chiral centers. These compounds are prepared by any means, including enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, chromatography, and the like.

The compounds described herein, and other related compounds having different substituents are synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001), and Green and Wuts, PROTECTIVE GROUPS IN ORGANIC SYNTHESIS 3^{rd} Ed., (Wiley 1999) (all of which are incorporated by reference for such disclosure). General methods for the preparation of compound as disclosed herein are modified by the use of appropriate reagents and conditions, for the introduction of the various moieties found in the formulae as provided herein. As a guide the following synthetic methods are utilized.

Compounds described herein are synthesized starting from compounds that are available from commercial sources or that are prepared using procedures outlined herein.

Formation of Covalent Linkages by Reaction of an Electrophile with a Nucleophile

The compounds described herein are modified using various electrophiles and/or nucleophiles to form new functional groups or substituents. Table A entitled “Examples of Covalent Linkages and Precursors Thereof” lists selected non-limiting examples of covalent linkages and precursor functional groups which yield the covalent linkages. Table A is used as guidance toward the variety of electrophiles and nucleophiles combinations available that provide covalent linakges. Precursor functional groups are shown as electrophilic groups and nucleophilic groups.

TABLE A
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

Use of Protecting Groups

In the reactions described, it is necessary to protect reactive functional groups, for example hydroxy, amino, imino, thio or carboxy groups, where these are desired in the final product, in order to avoid their unwanted participation in reactions. Protecting groups are used to block some or all of the reactive moieties and prevent such groups from participating in chemical reactions until the protective group is removed. In some embodiments it is contemplated that each protective group be removable by a different means. Protective groups that are cleaved under totally disparate reaction conditions fulfill the requirement of differential removal.

In some embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl in the presence of amines blocked with acid labile groups such as t-butyl carbamate or with carbamates that are both acid and base stable but hydrolytically removable.

In some embodiments carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in then presence of acid- and base-protecting groups since the former are stable and are subsequently removed by metal or pi-acid catalysts. For example, an allyl-blocked carboxylic acid is deprotected with a Pd⁰-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups are selected from:

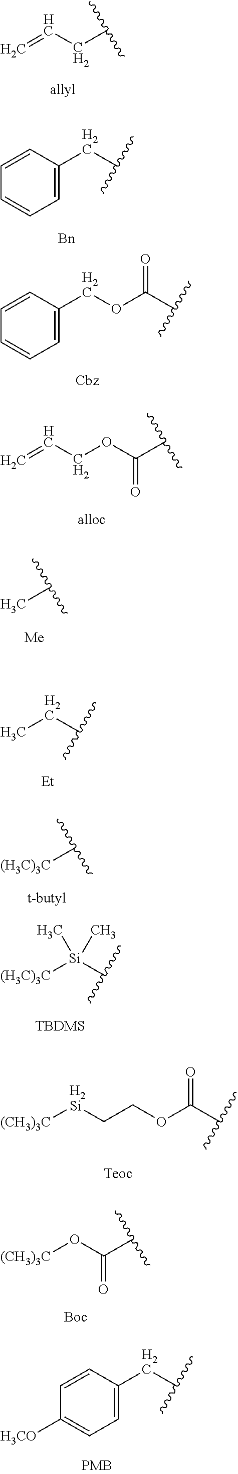

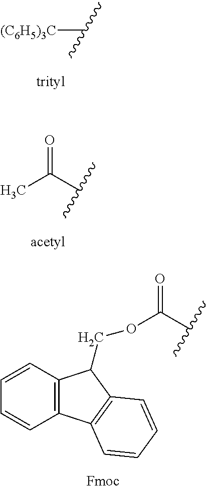

Other protecting groups, plus a detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure.

In specific embodiments, compounds described herein are prepared according to the process set forth in Scheme 1:

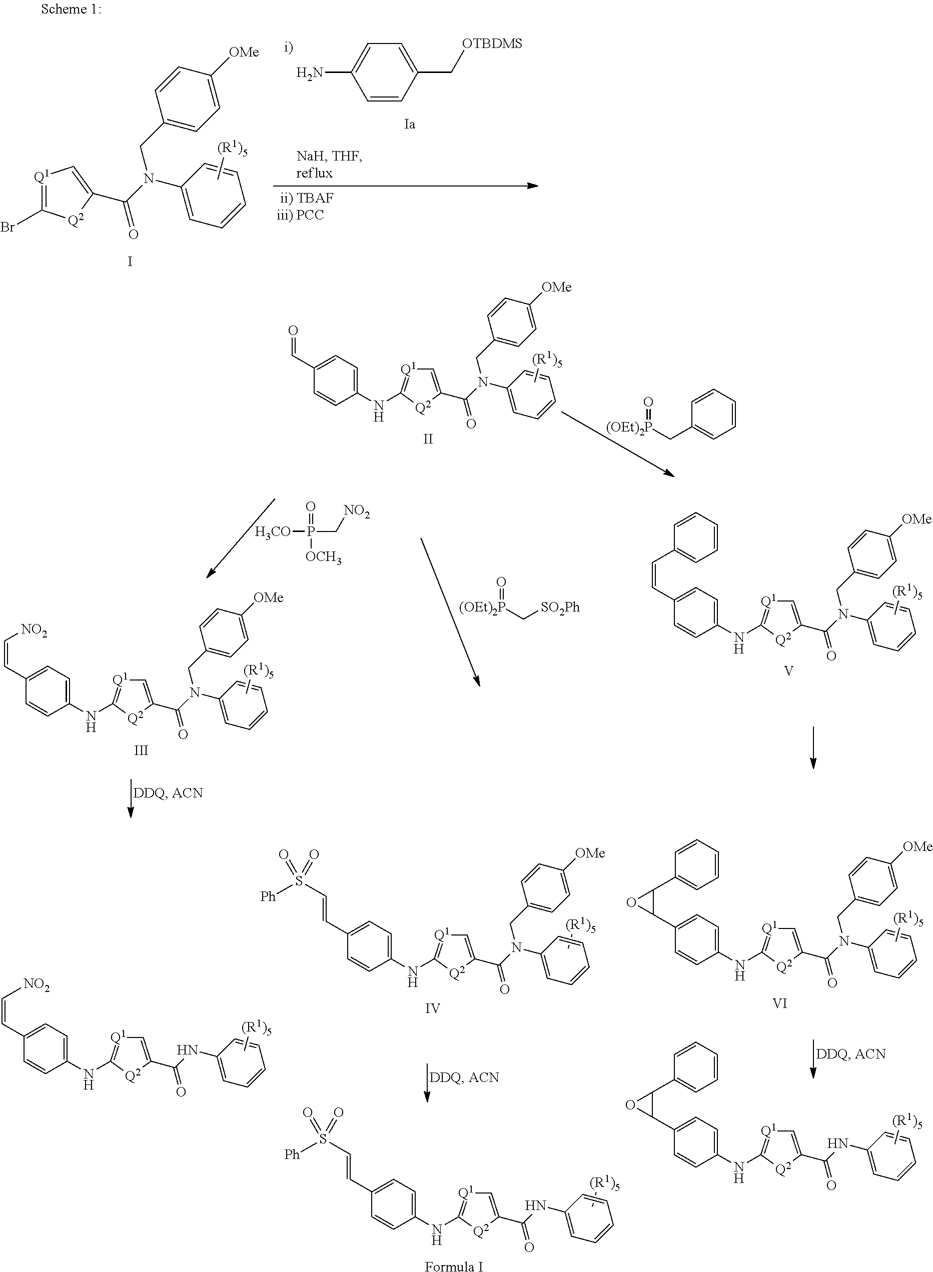

In certain embodiments, the synthesis begins with a compound of structure I. Such compounds are prepared in any suitable manner, e.g., as set forth U.S. Pat. Nos. 6,596,746, 7,112,599. In some embodiments, compounds of structure 1 are subject to a bromide displacement with an appropriate amine followed by removal of the silyl protecting group provides an alcohol. In certain embodiments, the alcohol is oxidized to an aldehyde using any suitable methods to provide a compound of structure III. In an alternative embodiment, the amine of structure Ia optionally contains a ketone group. In certain embodiments, the aldehyde of structure II is reacted with one of a variety of Wadsworth-Emmons reagents (See, e.g., U.S. Pat. No. 6,287,840) or are commercially available to provide compounds of structures IV, V and VI. In some embodiments, reactions with Wadsworth-Emmons reagents are carried out with a ketone moiety using suitable methods.

In certain embodiments, other amines of structure Ia (e.g., —NR¹-Aₙ₋₁C(═O)H, —NR¹-Aₙ₋₁C(═O)OH, —NR¹-Aₙ₋₁C(═O)Oalkyl, —NR¹-Aₙ₋₁CH₂OH) are utilized to synthesize compounds of structures IV, V or VI. Non limiting examples of amines of structure Ia that are useful in making compounds of structure II include:

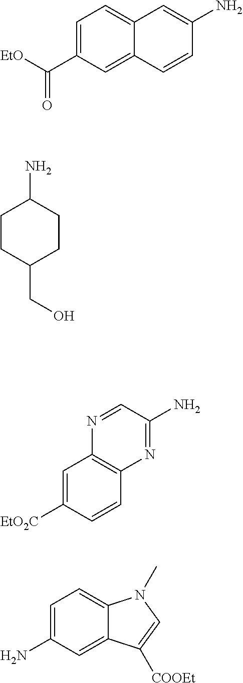

Depending on the structure of the amine 1a used in the synthesis, various methods for the protection/deprotection of moieties and/or the conversion of compounds of structure I to the aldehyde or ketone compounds of structure II are optionally utilized.

In one embodiment, a compound of structure V is converted to an epoxide compound of structure VI using any suitable method. In certain embodiments, a final step, deprotection of the amide protecting group under oxidative conditions affords compounds of Formula I.

In an alternative embodiment, the compounds described herein are prepared according to Scheme 2.

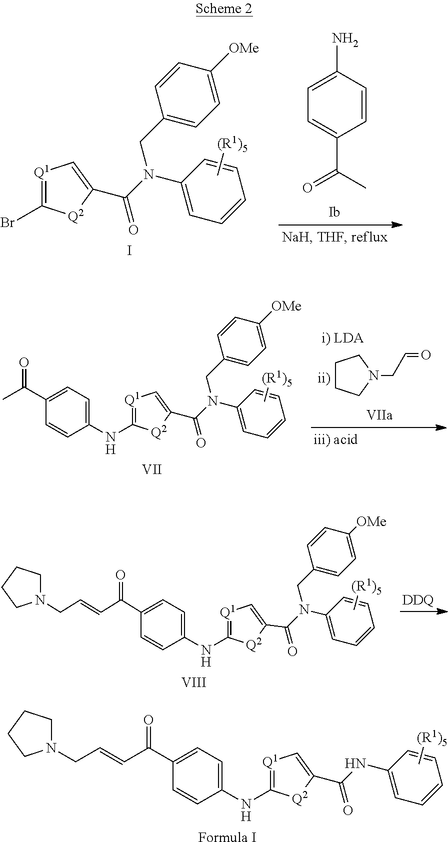

In some instances, the bromide in a compound of structure I is displaced in a reaction with an amine to provide a compound of structure VII. In some embodiments, an aldol reaction with an aldehyde of structure VIIa followed by a dehydration provides the α,β-unsaturated compound of structure VIII.

Aldehydes of structure VIIa are optionally prepared according to any suitable process. Non-limiting examples of aldehydes of structure VIIa that are optionally utilized for the synthesis of compound of structure VIII include R′₂N-alkyl-CHO, R′₃C-alkyl-CHO, R′₃CCHO, or the like (wherein R′ is selected from any suitable group, two R′ groups taken together form a substituted or unsubstituted ring) e.g.:

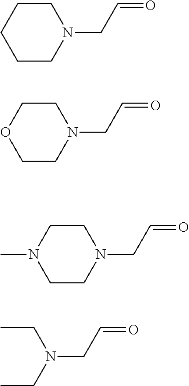

Other amines of structure Ib (e.g., —NR¹-Aₙ₋₁C(═O)H, —NR¹-Aₙ₋₁C(═O)OH, —NR¹-Aₙ₋₁C(═O)Oalkyl, —NR¹-Aₙ₋₁CH₂OH) are optionally utilized to synthesize the compounds of structures VII. Non limiting examples of amines of structure Ib that are useful in making compounds of structure VII include:

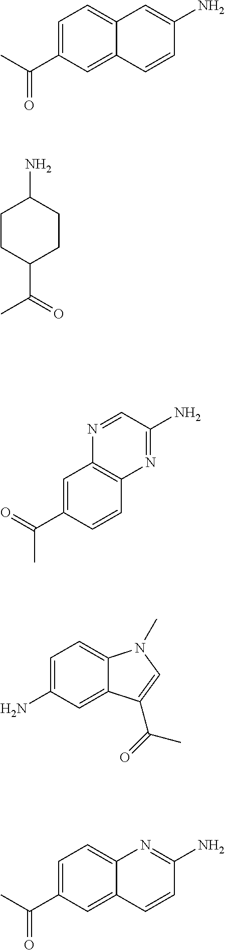

In another embodiment compounds of formula I are made according to Scheme 3.

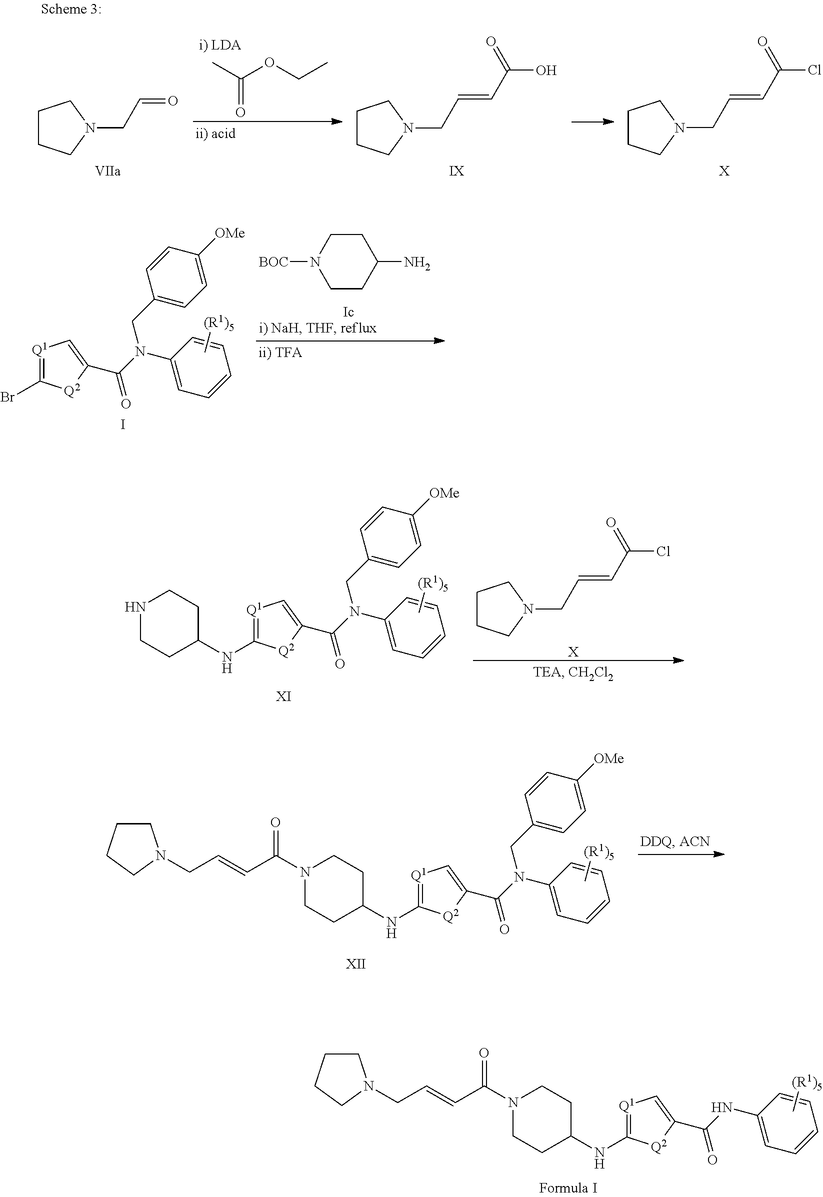

In some embodiments, an aldol reaction of an aldehyde of structure VIIa with ethyl acetate is followed by dehydration and ester hydrolysis under acidic conditions provides a compound of structure IX. In certain embodiments, the compound of structure IX is converted to an acid chloride of structure X.

In some embodiments, bromide displacement in the compound of structure I with an amine of structure Ic is followed by removal of the BOC protecting group provides a compound of structure XI. In some embodiments, reaction of a compound of structure XI with an acid chloride of structure X provides a compound of structure XII. In certain embodiments, deprotection of the amide in compound XII provides a compound of Formula I.

Other amines of structure Ic are optionally utilized to make the compounds of structures VII (e.g., NR¹Aₙ₋₁, wherein at least one of the A in Aₙ₋₁ comprises a primary or secondary amine). Non limiting examples of amines of structure Ic that are useful in making compounds of structure XII include:

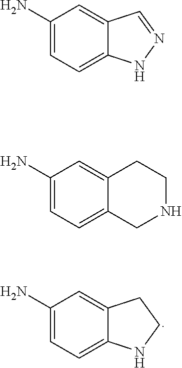

In one embodiment, shown in Scheme 4, the compound of structure XI reacts with an acyl halide of structure XIII to provide a compound of structure XIII. Deprotection of the amide bond in compound XIII affords a compound of Formula I

In one embodiment, shown in Scheme 4, the compound of structure XI reacts with an acyl halide of structure XIII to provide a compound of structure XIII. Deprotection of the amide bond in compound XIII affords a compound of Formula I.

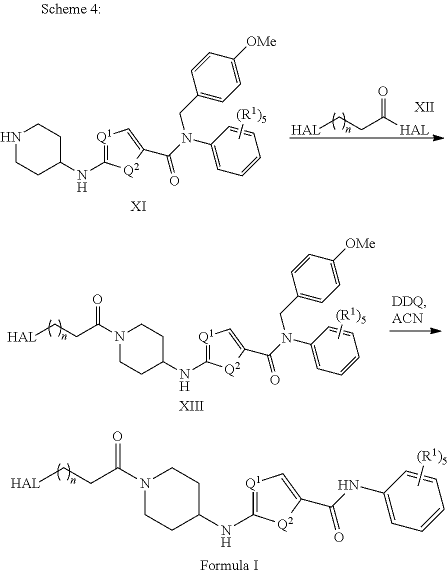

Acyl halides of formula XII that are used to synthesize compounds of formula I include, and are not limited to:

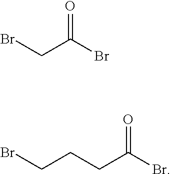

### GENERAL DEFINITIONS

The term “subject”, “patient” or “individual” are used interchangeably herein and refer to mammals and non-mammals, e.g., suffering from a disorder described herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” and other grammatical equivalents as used herein, include alleviating, inhibiting or reducing symptoms, reducing or inhibiting severity of, reducing incidence of, prophylactic treatment of, reducing or inhibiting recurrence of, preventing, delaying onset of, delaying recurrence of, abating or ameliorating a disease or condition symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit. By therapeutic benefit is meant eradication or amelioration of the underlying disorder being treated, and/or the eradication or amelioration of one or more of the physiological symptoms associated with the underlying disorder such that an improvement is observed in the patient.

The terms “prevent,” “preventing” or “prevention,” and other grammatical equivalents as used herein, include preventing additional symptoms, preventing the underlying metabolic causes of symptoms, inhibiting the disease or condition, e.g., arresting the development of the disease or condition and are intended to include prophylaxis. The terms further include achieving a prophylactic benefit. For prophylactic benefit, the compositions are optionally administered to a patient at risk of developing a particular disease, to a patient reporting one or more of the physiological symptoms of a disease, or to a patient at risk of reoccurrence of the disease.

Where combination treatments or prevention methods are contemplated, it is not intended that the agents described herein be limited by the particular nature of the combination. For example, the agents described herein are optionally administered in combination as simple mixtures as well as chemical hybrids. An example of the latter is where the agent is covalently linked to a targeting carrier or to an active pharmaceutical. Covalent binding can be accomplished in many ways, such as, though not limited to, the use of a commercially available cross-linking agent. Furthermore, combination treatments are optionally administered separately or concomitantly.

As used herein, the terms “pharmaceutical combination”, “administering an additional therapy”, “administering an additional therapeutic agent” and the like refer to a pharmaceutical therapy resulting from the mixing or combining of more than one active ingredient and includes both fixed and non-fixed combinations of the active ingredients. The term “fixed combination” means that at least one of the agents described herein, and at least one co-agent, are both administered to a patient simultaneously in the form of a single entity or dosage. The term “non-fixed combination” means that at least one of the agents described herein, and at least one co-agent, are administered to a patient as separate entities either simultaneously, concurrently or sequentially with variable intervening time limits, wherein such administration provides effective levels of the two or more agents in the body of the patient. In some instances, the co-agent is administered once or for a period of time, after which the agent is administered once or over a period of time. In other instances, the co-agent is administered for a period of time, after which, a therapy involving the administration of both the co-agent and the agent are administered. In still other embodiments, the agent is administered once or over a period of time, after which, the co-agent is administered once or over a period of time. These also apply to cocktail therapies, e.g. the administration of three or more active ingredients.

As used herein, the terms “co-administration”, “administered in combination with” and their grammatical equivalents are meant to encompass administration of the selected therapeutic agents to a single patient, and are intended to include treatment regimens in which the agents are administered by the same or different route of administration or at the same or different times. In some embodiments the agents described herein will be co-administered with other agents. These terms encompass administration of two or more agents to an animal so that both agents and/or their metabolites are present in the animal at the same time. They include simultaneous administration in separate compositions, administration at different times in separate compositions, and/or administration in a composition in which both agents are present. Thus, in some embodiments, the agents described herein and the other agent(s) are administered in a single composition. In some embodiments, the agents described herein and the other agent(s) are admixed in the composition.

The terms “effective amount” or “therapeutically effective amount” as used herein, refer to a sufficient amount of at least one agent being administered which achieve a desired result, e.g., to relieve to some extent one or more symptoms of a disease or condition being treated. In certain instances, the result is a reduction and/or alleviation of the signs, symptoms, or causes of a disease, or any other desired alteration of a biological system. In certain instances, an “effective amount” for therapeutic uses is the amount of the composition comprising an agent as set forth herein required to provide a clinically significant decrease in a disease. An appropriate “effective” amount in any individual case is determined using any suitable technique, such as a dose escalation study.

The terms “administer,” “administering”, “administration,” and the like, as used herein, refer to the methods that may be used to enable delivery of agents or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Administration techniques that are optionally employed with the agents and methods described herein are found in sources e.g., Goodman and Gilman, The Pharmacological Basis of Therapeutics, current ed.; Pergamon; and Remington's, Pharmaceutical Sciences (current edition), Mack Publishing Co., Easton, Pa. In certain embodiments, the agents and compositions described herein are administered orally.

The term “pharmaceutically acceptable” as used herein, refers to a material that does not abrogate the biological activity or properties of the agents described herein, and is relatively nontoxic (i.e., the toxicity of the material significantly outweighs the benefit of the material). In some instances, a pharmaceutically acceptable material may be administered to an individual without causing significant undesirable biological effects or significantly interacting in a deleterious manner with any of the components of the composition in which it is contained.

The term “carrier” as used herein, refers to relatively nontoxic chemical agents that, in certain instances, facilitate the incorporation of an agent into cells or tissues.

In various embodiments, pharmaceutically acceptable salts described herein include, by way of non-limiting example, a nitrate, chloride, bromide, phosphate, sulfate, acetate, hexafluorophosphate, citrate, gluconate, benzoate, propionate, butyrate, sulfosalicylate, maleate, laurate, malate, fumarate, succinate, tartrate, amsonate, pamoate, p-toluenenesulfonate, mesylate and the like. Furthermore, pharmaceutically acceptable salts include, by way of non-limiting example, alkaline earth metal salts (e.g., calcium or magnesium), alkali metal salts (e.g., sodium or potassium), ammonium salts and the like.

The term “optionally substituted” or “substituted” means that the referenced group substituted with one or more additional group(s). In certain embodiments, the one or more additional group(s) are individually and independently selected from amide, ester, alkyl, cycloalkyl, heteroalkyl, aryl, heteroaryl, heteroalicyclic, hydroxy, alkoxy, aryloxy, alkylthio, arylthio, alkylsulfoxide, arylsulfoxide, ester, alkylsulfone, arylsulfone, cyano, halo, alkoyl, alkoyloxo, isocyanato, thiocyanato, isothiocyanato, nitro, haloalkyl, haloalkoxy, fluoroalkyl, amino, alkyl-amino, dialkyl-amino, amido.

An “alkyl” group refers to an aliphatic hydrocarbon group. Reference to an alkyl group includes “saturated alkyl” and/or “unsaturated alkyl”. The alkyl group, whether saturated or unsaturated, includes branched, straight chain, or cyclic groups. By way of example only, alkyl includes methyl, ethyl, propyl, iso-propyl, n-butyl, iso-butyl, sec-butyl, t-butyl, pentyl, iso-pentyl, neo-pentyl, and hexyl. In some embodiments, alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. A “lower alkyl” is a C₁-C₆ alkyl. A “heteroalkyl” group substitutes any one of the carbons of the alkyl group with a heteroatom having the appropriate number of hydrogen atoms attached (e.g., a CH₂ group to an NH group or an O group).

An “alkoxy” group refers to a (alkyl)O— group, where alkyl is as defined herein.

The term “alkylamine” refers to the —N(alkyl)ₓH_{y} group, wherein alkyl is as defined herein and x and y are selected from the group x=1, y=1 and x=2, y=0. When x=2, the alkyl groups, taken together with the nitrogen to which they are attached, optionally form a cyclic ring system.

An “amide” is a chemical moiety with formula —C(O)NHR or —NHC(O)R, where R is selected from alkyl, cycloalkyl, aryl, heteroaryl (bonded through a ring carbon) and heteroalicyclic (bonded through a ring carbon).

The term “ester” refers to a chemical moiety with formula —C(═O)OR, where R is selected from the group consisting of alkyl, cycloalkyl, aryl, heteroaryl and heteroalicyclic.

As used herein, the term “aryl” refers to an aromatic ring wherein each of the atoms forming the ring is a carbon atom. Aryl rings disclosed herein include rings having five, six, seven, eight, nine, or more than nine carbon atoms. Aryl groups are optionally substituted. Examples of aryl groups include, but are not limited to phenyl, and naphthalenyl.

The term “cycloalkyl” refers to a monocyclic or polycyclic non-aromatic radical, wherein each of the atoms forming the ring (i.e. skeletal atoms) is a carbon atom. In various embodiments, cycloalkyls are saturated, or partially unsaturated. In some embodiments, cycloalkyls are fused with an aromatic ring. Cycloalkyl groups include groups having from 3 to 10 ring atoms. Illustrative examples of cycloalkyl groups include, but are not limited to, the following moieties:

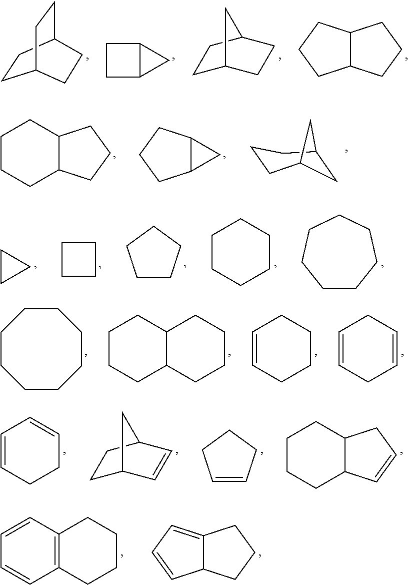

and the like. Monocyclic cycloalkyls include, but are not limited to, cyclopropyl, cyclobutyl, cyclopentyl, cyclohexyl, cycloheptyl, and cyclooctyl.

The term “heterocyclo” refers to heteroaromatic and heteroalicyclic groups containing one to four ring heteroatoms each selected from O, S and N. In certain instances, each heterocyclic group has from 4 to 10 atoms in its ring system, and with the proviso that the ring of said group does not contain two adjacent O or S atoms. Non-aromatic heterocyclic groups include groups having 3 atoms in their ring system, but aromatic heterocyclic groups must have at least 5 atoms in their ring system. The heterocyclic groups include benzo-fused ring systems. An example of a 3-membered heterocyclic group is aziridinyl (derived from aziridine). An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. In certain embodiments, heteroaryl groups are monocyclic or polycyclic. Illustrative examples of heteroaryl groups include the following moieties:

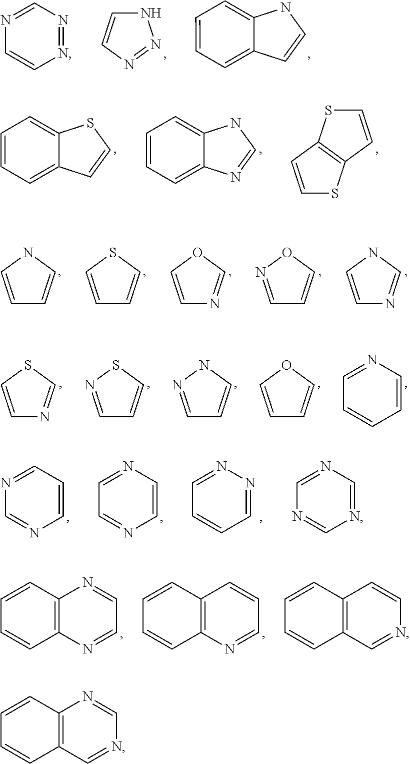

and the like.

A “heteroalicyclic” group or “heterocyclo” group refers to a cycloalkyl group, wherein at least one skeletal ring atom is a heteroatom selected from nitrogen, oxygen and sulfur. In various embodiments, the radicals are with an aryl or heteroaryl. Illustrative examples of heterocyclo groups, also referred to as non-aromatic heterocycles, include:

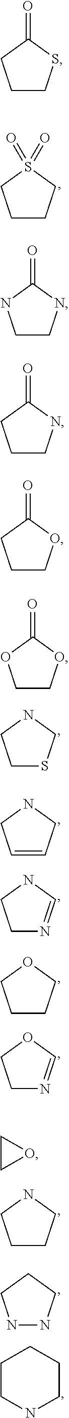

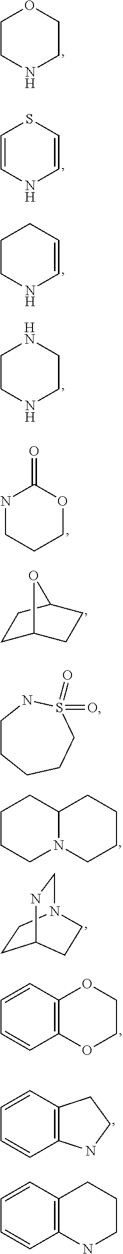

and the like. The term heteroalicyclic also includes all ring forms of the carbohydrates, including but not limited to the monosaccharides, the disaccharides and the oligosaccharides.

The term “halo” or, alternatively, “halogen” means fluoro, chloro, bromo and iodo.

The terms “haloalkyl,” and “haloalkoxy” include alkyl and alkoxy structures that are substituted with one or more halogens. In embodiments, where more than one halogen is included in the group, the halogens are the same or they are different. The terms “fluoroalkyl” and “fluoroalkoxy” include haloalkyl and haloalkoxy groups, respectively, in which the halo is fluorine.

The term “heteroalkyl” include optionally substituted alkyl, alkenyl and alkynyl radicals which have one or more skeletal chain atoms selected from an atom other than carbon, e.g., oxygen, nitrogen, sulfur, phosphorus, silicon, or combinations thereof. In certain embodiments, the heteroatom(s) is placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In some embodiments, up to two heteroatoms are consecutive, such as, by way of example, —CH₂—NH—OCH₃ and —CH₂—O—Si(CH₃)₃.

A “cyano” group refers to a —CN group.

An “isocyanato” group refers to a —NCO group.

A “thiocyanato” group refers to a —CNS group.

An “isothiocyanato” group refers to a —NCS group.

“Alkoyloxy” refers to a RC(═O)O— group.

“Alkoyl” refers to a RC(═O)— group.

### Methods

In certain embodiments, provided herein is a method of inhibiting, reducing the activity of, knocking down, or modulating the activity of a kinase by contacting the kinase with an effective amount of any compound described herein. In some embodiments, the kinase is a cysteine containing kinase. In certain embodiments, the method provides a method of irreversibly inhibiting, reducing the activity of, knocking down, or modulating the activity of a kinase by contacting the kinase with an effective amount of any compound described herein. In specific embodiments, the kinase comprises a cysteine residue near an ATP-binding site of the kinase. In more specific embodiments, the cysteine residue is in close spatial proximity to an ATP-binding site of the kinase. In some embodiments, the kinase comprising a cysteine residue near an ATP-binding site includes, by way of non-limiting example, BTK, BMX, TEC, TXK, ITK, EGFR, ErbB2, ErbB4, JAK3, BLK, or the like. In specific embodiments, the kinase is BTK or TEC. In some embodiments, the kinase is a tyrosine kinase. In more specific embodiments, the tyrosine kinase is a Tec family kinase. In some specific embodiments, the tyrosine kinase comprises a cysteine residue near an ATP-binding site of the tyrosine kinase. In more specific embodiments, the cysteine residue is in close spatial proximity to an ATP-binding site of the tyrosine kinase. In some embodiments, compounds described herein are also utilized in methods of inhibiting, reducing the activity of, or modulating the activity of ABL or SRC. In some embodiments, the method is performed in vitro, or in vivo. In some embodiments, when performed in vivo, the individual to which the compound is administered has been diagnosed with a disease or disorder disclosed herein (e.g., a kinase mediated disorder disclosed herein).

In some embodiments, provided herein is a method of binding a cysteine containing kinase to a compound of Formula I comprising contacting the kinase with the compound of Formula I. In some embodiments, the process of binding the compound to the kinase comprises forming a covalent bond between the kinase and the compound of Formula I. In specific embodiments, the process of binding is an irreversible process. In specific embodiments, the kinase comprises a cysteine residue near an ATP-binding site of the kinase. In more specific embodiments, the cysteine residue is in close spatial proximity to an ATP-binding site of the kinase. In some embodiments, the kinase comprising a cysteine residue near an ATP-binding site includes, by way of non-limiting example, BTK, BMX, TEC, TXK, ITK, EGFR, ErbB2, ErbB4, JAK3, BLK, or the like. In specific embodiments, the kinase is BTK or TEC. In some embodiments, the kinase is a tyrosine kinase. In more specific embodiments, the tyrosine kinase is a Tec family kinase. In some specific embodiments, the tyrosine kinase comprises a cysteine residue near an ATP-binding site of the tyrosine kinase. In more specific embodiments, the cysteine residue is in close spatial proximity to an ATP-binding site of the tyrosine kinase. In some embodiments, the method is performed in vitro, or in vivo. In some embodiments, when performed in vivo, the individual to which the compound is administered has been diagnosed with a disease or disorder disclosed herein (e.g., a kinase mediated disorder disclosed herein).

In some embodiments, provided herein is a method of decreasing the dose necessary of a therapeutic agent to treat a kinase mediated disorder in an individual in need thereof by replacing a Compound 100 treatment with a treatment comprising administering to the individual a therapeutically effective amount of a substituted or modified Compound 100 compound described herein. Thus, in some embodiments, provided herein is also a method of treating a disease or disorder mediated by a kinase by administering to an individual in need thereof a therapeutically effective amount of a compound described herein, wherein the therapeutically effective amount is less than a therapeutically effective amount of Compound 100 (by weight and/or molar amount). In specific embodiments, the kinase comprises a cysteine residue near an ATP-binding site of the kinase. In more specific embodiments, the cysteine residue is in close spatial proximity to an ATP-binding site of the kinase. In some embodiments, the kinase comprising a cysteine residue near an ATP-binding site includes, by way of non-limiting example, BTK, BMX, TEC, TXK, ITK, EGFR, ErbB2, ErbB4, JAK3, BLK, or the like. In specific embodiments, the kinase is BTK or TEC. In some embodiments, the kinase is a tyrosine kinase. In more specific embodiments, the tyrosine kinase is a Tec family kinase. In some specific embodiments, the tyrosine kinase comprises a cysteine residue near an ATP-binding site of the tyrosine kinase. In more specific embodiments, the cysteine residue is in close spatial proximity to an ATP-binding site of the tyrosine kinase. In some embodiments, the individual to which the compound is administered has been diagnosed with a disease or disorder disclosed herein (e.g., a kinase mediated disorder disclosed herein).

In certain embodiments, provided herein is a method of administering an effective amount of any compound described herein to an individual in need thereof for the treatment of a disease or disorder mediated by a kinase. In some embodiments, the kinase is a cysteine containing kinase. In specific embodiments, the kinase comprises a cysteine residue near an ATP-binding site of the kinase. In more specific embodiments, the cysteine residue is in close spatial proximity to an ATP-binding site of the kinase. In some embodiments, the kinase comprising a cysteine residue near an ATP-binding site includes, by way of non-limiting example, BTK, BMX, TEC, TXK, ITK, EGFR, ErbB2, ErbB4, JAK3, BLK, or the like. In specific embodiments, the kinase is BTK or TEC. In some embodiments, the kinase is a tyrosine kinase. In more specific embodiments, the tyrosine kinase is a Tec family kinase. In some specific embodiments, the tyrosine kinase comprises a cysteine residue near an ATP-binding site of the tyrosine kinase. In more specific embodiments, the cysteine residue is in close spatial proximity to an ATP-binding site of the tyrosine kinase. In some embodiments, compounds described herein are also utilized in methods treating diseases or disorders mediated by ABL or SRC.

Kinases play critical roles in signaling pathways controlling fundamental cellular processes such as proliferation, differentiation, and death (apoptosis). Abnormal kinase activity is implicated in a wide range of diseases, including multiple cancers and autoimmune and inflammatory diseases.

Diseases mediated by receptor kinase activity include, but are not limited to, diseases characterized in part by abnormal levels of cytokines (i.e., inflammation), cell proliferation (e.g. cancer), programmed cell death (apoptosis), cell migration and invasion, and angiogenesis associated with tumor growth.

In some embodiments, disclosed herein are methods and compositions for the modulation, and treatment of immune, inflammatory, respiratory, autoimmune, cardiovascular, neuronal, ischemic, hematological and proliferative disorders. In certain embodiments, such disorders are treated by administering a therapeutically effective amount of a compound described herein to an individual in need thereof (e.g., an individual diagnosed with one or more of such disorders).

Immune disorders include, but are not limited to, chronic inflammatory diseases and autoimmune disorders, such as Crohn's disease, reactive arthritis, including Lyme disease, systemic lupus erythematosus (SLE), insulin-dependent diabetes, organ-specific auto immunity, Hashimoto's thyroiditis and Grave's disease, contact dermatitis, psoriasis, organ transplant rejection, graft rejection, graft versus host disease, sarcoidosis, atopic conditions, gastrointestinal allergies, including food allergies, pancreatitis, inflammatory bowel disease, eosinophilia, conjunctivitis, glomerular nephritis, multiple vasculitides, myasthenia gravis, certain pathogen susceptibilities such as helminthic infections (e.g., leishmaniasis), certain viral infections, including HIV, and bacterial infections, including tuberculosis and lepromatous leprosy.

Respiratory disorders include, but are not limited to, apnea, asthma, particularly bronchial asthma, allergy, including allergic rhinitis, berillium disease, bronchiectasis, bronchitis, bronchopneumonia, cystic fibrosis, diphtheria, dyspnea, emphysema, chronic obstructive pulmonary disease, allergic bronchopulmonary aspergillosis, pneumonia, acute pulmonary edema, pertussis, pharyngitis, atelectasis, Wegener's granulomatosis, Legionnaires disease, pleurisy, rheumatic fever, and sinusitis.

Hematologic disorders include but are not limited to anemias including sickle cell and hemolytic anemia, erythrocytosis, hemophilias including types A and B, leukemias, thalassemias, spherocytosis, Von Willebrand disease, chronic granulomatous disease, glucose-6-phosphate dehydrogenase deficiency, thrombosis, clotting factor abnormalities and deficiencies including factor VIII and IX deficiencies, hemarthrosis, hematemesis, hematomas, hematuria, hemochromatosis, hemoglobinuria, hemolytic-uremic syndrome, thrombocytopenias including HIV-associated thrombocytopenia, hemorrhagic telangiectasia, idiopathic thrombocytopenic purpura, thrombotic microangiopathy, hemosiderosis.

Proliferative disorders include and are not limited to cancer, including breast and ovarian cancers, epithelial cancers such as gastric adenocarcinoma, prostate cancer, lung cancer, head and neck cancer, bladder cancer, melanoma, oesophageal cancer, lymphoma, including B-cell and Hodgkins lymphoma, brain tumors, colorectal cancer, renal cancer, squamous cell cancer, glioblastoma, Kaposi's sarcoma, multiple myeloma, and leukemia (e.g. myeloid, chronic myeloid, acute lymphoblastic, chronic lymphoblastic, and other leukemias and hematological cancers).

Neuronal disorders include and are not limited to Alzheimers disease, Parkinson's disease, dementia, Huntington's disease, multiple sclerosis, neuronal ceroid lipofuscinosis, autism and epilepsy.

Ischemic disorders include and are not limited to liver ischemia, myocardial infarction and reperfusion injury.

Cardiovascular disorders include heart failure, hypertension, atrial fibrillation, dilated cardiomyopathy, idiopathic cardiomyopathy, or angina.

Bruton's Tyrosine Kinase (Btk) is a member of the Tec family of tyrosine kinases, and is a critical regulator of early B-cell development as well as mature B-cell activation, signaling and survival. B-cell signaling through the B-cell receptor (BCR) leads to a wide range of biological outputs. Aberrant BCR-mediated signaling can cause disregulated B-cell proliferation and/or the formation of pathogenic auto-antibodies leading to multiple autoimmune and/or inflammatory diseases. Mutation of Btk in humans results in X-linked agammaglobulinaemia (XLA). This disease is associated with the impaired maturation of B-cells, diminished immunoglobulin production, comprised T-cell-independent immune responses and marked attenuation of the sustained calcium sign upon BCR stimulation.

Inhibition of Btk activity is useful for the treatment of autoimmune and/or inflammatory diseases such as: SLE, rheumatoid arthritis, multiple vasculitides, idiopathic thrombocytopenic purpura (ITP), myasthenia gravis, and asthma. In addition, Btk has been reported to play a role in apoptosis; inhibition of Btk activity is useful for the treatment of B-cell lymphoma and leukemia.

Human epidermal growth factor (EGF) is a 53 amino acid, single-chain polypeptide (Mr 6216 daltons), which exerts biologic effects by binding to a specific cell membrane epidermal growth factor receptor (EGFR/ErbB-1). In certain instances, EGFR mediated disorders include cancers, such as, by way of non-limiting example, breast cancer, prostate cancer, lung cancer, head and neck cancer, bladder cancer, melanoma, and brain tumors (Khazaie, K., et al. R. B. Cancer & Metastasis Reviews 1993, 12, 255).

HER4/Erb4 is a receptor protein tyrosine kinase belonging to the ErbB family. Increased ErbB4 expression closely correlates with certain carcinomas of epithelial origin, including breast adenocarcinomas (Plowman et al., Proc. Natl. Acad. Sci. USA 90:1746-1750 [1993]; Plowman et al., Nature 366:473-475 [1993]). Other members of the ErbB family of receptor tyrosine kinases include: epidermal growth factor receptor (EGFR), ErbB2 (HER2/neu), and ErbB3 (HER3). HER4 acts, in the absence of HER2, as a mediator of antiproliferative and differentiative response in human breast cancer cell lines. (Sartor et al., Mol. Cell. Biol. 21:4265-75 (2001). In some instances, Erb4/Erb2 mediated disorders include epithelial malignancies such as breast cancer.

Smooth muscle cells from a variety of organs such as the heart and the urinary bladder possess EGF receptors. Various EGF ligands act as potent mitogens and stimulate proliferation of smooth muscle cells often resulting in thickening of the wall and ultimately stenosis. EGFR mediated disorders include disorders caused by excessive proliferation of vascular smooth muscle cells (VSMC) such as vascular stenosis, restenosis resulting from angioplasty or surgery or stent implants, atherosclerosis, transplant atherosclerosis and hypertension (reviewed in Casterella and Teirstein, Cardiol. Rev. 7: 219-231 [1999]; Andres, Int. J. Mol. Med. 2: 81-89 [1998]; and Rosanio at al, Thromb. Haemost. 82 [suppl 1]: 164-170 [1999]). Excessive proliferation of VSMC can cause decreased blood supply to tissues and may also cause necrosis and/or inflammatory response leading to severe damage. For example, myocardial infarction occurs as a result of lack of oxygen and local death of heart muscle tissues.

EGF receptor mediated excessive proliferation of urinary bladder smooth muscle cells causes obstruction and hyperplasia of the bladder. Infantile hypertrophic pyloric stenosis (IHPS), which causes functional obstruction of the pyloric canal with hypertrophy and hyperplasia of the pyloric smooth muscle cells, may be mediated by EGFR (Due and Pun, Pediatr. Res. 45: 853-857 [1999]).

The obstructive airway diseases are yet another group of diseases with underlying pathology involving EFG receptor mediated smooth muscle cell proliferation. One example of this group is asthma which manifests in airway inflammation and bronchoconstriction.

The Src-family of tyrosine kinase plays a critical role in blood cell function. Many members of the Src-family of tyrosine kinases are found exclusively or primarily in blood cells, and inhibitors of Src kinases block leukemic cell growth (Corey et al., Leukemia. 1999; 13(6):855-61). Disorders mediated by Src tyrosine kinase may also include, by way of non-limiting example, hematologic tumors, and solid tumors.

Excessive tyrosine kinase activity is also associated with inflammatory and autoimmune diseases. In some instances, Src (e.g., Lyn, Hck, Lck, Fgr, and Blk) mediated disorders may include, by way of non-limiting example, allergic diseases, autoimmunity, and transplantation rejection.

It is also believed that the Aβ peptide in senile plaques activates Src tyrosine kinases. In certain instances, Src mediated disorders may include, by way of non-limiting example, CNS disorders including, but not limited to, Parkinsons Disease and chronic pain. Increased neuronal Src kinase activity induces epileptiform discharges. The frequency of the epileptiform discharges is decreased by the addition of an inhibitor of the Src family of tyrosine kinases. Additional Src mediated disorders include epilepsy and other disorders related to NMDA receptor function (Sanna et al., Proc Natl Acad Sci USA. 2000, 18; 97(15):8653-7).

Herpesviridae, papovaviridae, and retroviridae interact with non-receptor tyrosine kinases and use them as signaling intermediates. For example, the HIV-1 Nef protein interacts with members of the Src family of tyrosine kinases. In some instances, the Src tyrosine kinases mediate diseases caused by viral proteins such as polyomavirus middle-T antigens, Epstein-Barr virus LMP2A, and herpesvirus saimiri Tip (Dunant and Ballmer-Hofer, Cell Signal. 1997; 9(6):385-93).

The Janus kinases (JAK1, JAK2 and JAK3) are tyrosine kinases that play a critical role in cytokine signaling and are implicated in the mediation of many abnormal immune responses such as allergies, asthma, autoimmune diseases such as transplant rejection, rheumatoid arthritis, amyotrophic lateral sclerosis and multiple sclerosis, solid and hematologic malignancies such as leukemias and lymphomas, and proliferative disorders such as erythrocytosis (Frank Mol. Med. 5: 432 456 (1999) & Seidel, et al, Oncogene 19: 2645 2656 (2000)).

Administration of a compound described herein is achieved in any suitable manner including, by way of non-limiting example, by oral, parenteral (e.g., intravenous, subcutaneous, intramuscular), intranasal, buccal, topical, rectal, or transdermal administration routes.

In certain embodiments, a compound or a composition comprising a compound described herein is administered for prophylactic and/or therapeutic treatments. In therapeutic applications, the compositions are administered to an individual already suffering from a disease or condition, in an amount sufficient to cure or at least partially arrest the symptoms of the disease or condition. In various instances, amounts effective for this use depend on the severity and course of the disease or condition, previous therapy, the individual's health status, weight, and response to the drugs, and the judgment of the treating physician.

In prophylactic applications, compounds or compositions containing compounds described herein are administered to an individual susceptible to or otherwise at risk of a particular disease, disorder or condition. In certain embodiments of this use, the precise amounts of compound administered depend on the individual's state of health, weight, and the like. Furthermore, in some instances, when a compound or composition described herein is administered to an individual, effective amounts for this use depend on the severity and course of the disease, disorder or condition, previous therapy, the individual's health status and response to the drugs, and the judgment of the treating physician.

In certain instances, wherein following administration of a selected dose of a compound or composition described herein, an individual's condition does not improve, upon the doctor's discretion the administration of a compound or composition described herein is optionally administered chronically, that is, for an extended period of time, including throughout the duration of the individual's life in order to ameliorate or otherwise control or limit the symptoms of the individual's disorder, disease or condition.

In certain embodiments, an effective amount of a given agent varies depending upon one or more of a number of factors such as the particular compound, disease or condition and its severity, the identity (e.g., weight) of the subject or host in need of treatment, and is determined according to the particular circumstances surrounding the case, including, e.g., the specific agent being administered, the route of administration, the condition being treated, and the subject or host being treated. In some embodiments, doses administered include those up to the maximum tolerable dose. In certain embodiments, about 0.02-5000 mg per day, from about 1-1500 mg per day, about 1 to about 100 mg/day, about 1 to about 50 mg/day, or about 1 to about 30 mg/day, or about 5 to about 25 mg/day of a compound described herein is administered. In various embodiments, the desired dose is conveniently be presented in a single dose or in divided doses administered simultaneously (or over a short period of time) or at appropriate intervals, for example as two, three, four or more sub-doses per day.

In certain instances, there are a large number of variables in regard to an individual treatment regime, and considerable excursions from these recommended values are considered within the scope described herein. Dosages described herein are optionally altered depending on a number of variables such as, by way of non-limiting example, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are optionally determined by pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. In certain embodiments, data obtained from cell culture assays and animal studies are used in formulating a range of dosage for use in human. In specific embodiments, the dosage of compounds described herein lies within a range of circulating concentrations that include the ED₅₀ with minimal toxicity. The dosage optionally varies within this range depending upon the dosage form employed and the route of administration utilized.

### Combinations

In certain instances, provided herein are combination compositions and/or therapies comprising a compound of any of Formulas I-V and an additional therapeutic agent. In specific embodiments, the additional therapeutic agent is an anti-cancer agent, an anti-inflammatory agent, or an immunosuppressant.

In some embodiments, the particular choice of compounds depends upon the diagnosis of the attending physicians and their judgment of the condition of the individual and the appropriate treatment protocol. The compounds are optionally administered concurrently (e.g., simultaneously, essentially simultaneously or within the same treatment protocol) or sequentially, depending upon the nature of the disease, disorder, or condition, the condition of the individual, and the actual choice of compounds used. In certain instances, the determination of the order of administration, and the number of repetitions of administration of each therapeutic agent during a treatment protocol, is based on an evaluation of the disease being treated and the condition of the individual.

In some embodiments, therapeutically-effective dosages vary when the drugs are used in treatment combinations. Methods for experimentally determining therapeutically-effective dosages of drugs and other agents for use in combination treatment regimens are described in the literature.

In some embodiments of the combination therapies described herein, dosages of the co-administered compounds vary depending on the type of co-drug employed, on the specific drug employed, on the disease or condition being treated and so forth. In addition, when co-administered with one or more biologically active agents, the compound provided herein is optionally administered either simultaneously with the biologically active agent(s), or sequentially. In certain instances, if administered sequentially, the attending physician will decide on the appropriate sequence of therapeutic compound described herein in combination with the additional therapeutic agent.

The multiple therapeutic agents (at least one of which is a therapeutic compound described herein) are optionally administered in any order or even simultaneously. If simultaneously, the multiple therapeutic agents are optionally provided in a single, unified form, or in multiple forms (by way of example only, either as a single pill or as two separate pills). In certain instances, one of the therapeutic agents is optionally given in multiple doses. In other instances, both are optionally given as multiple doses. If not simultaneous, the timing between the multiple doses is any suitable timing, e.g, from more than zero weeks to less than four weeks. In some embodiments, the additional therapeutic agent is utilized to achieve remission (partial or complete) of a cancer, whereupon the therapeutic agent described herein (e.g., a compound of any one of Formulas I-V) is subsequently administered. In addition, the combination methods, compositions and formulations are not to be limited to the use of only two agents; the use of multiple therapeutic combinations are also envisioned (including two or more compounds described herein).

In certain embodiments, a dosage regimen to treat, prevent, or ameliorate the condition(s) for which relief is sought, is modified in accordance with a variety of factors. These factors include the disorder from which the subject suffers, as well as the age, weight, sex, diet, and medical condition of the subject. Thus, in various embodiments, the dosage regimen actually employed varies and deviates from the dosage regimens set forth herein.

In some embodiments, the pharmaceutical agents which make up the combination therapy disclosed herein are provided in a combined dosage form or in separate dosage forms intended for substantially simultaneous administration. In certain embodiments, the pharmaceutical agents that make up the combination therapy are administered sequentially, with either therapeutic compound being administered by a regimen calling for two-step administration. In some embodiments, two-step administration regimen calls for sequential administration of the active agents or spaced-apart administration of the separate active agents. In certain embodiments, the time period between the multiple administration steps varies, by way of non-limiting example, from a few minutes to several hours, depending upon the properties of each pharmaceutical agent, such as potency, solubility, bioavailability, plasma half-life and kinetic profile of the pharmaceutical agent.

In certain embodiments, therapeutic agents are combined with or utilized in combination with one or more of the following therapeutic agents in any combination: immunosuppressants or anti-cancer therapies (e.g., radiation, surgery or anti-cancer agents).

In some embodiments, the additional therapeutic agent is an anti-inflammatory agent. Specific anti-inflammatory agents include, by way of non-limiting example, steroids and NSAIDs. Non-steroidal anti-inflammatory drugs (NSAIDs) include, by way of non-limiting example, salicylates, amoxiprin, benorylate, choline magnesium salicylate, diflunisal, ethenzamide, faislamine, methyl salicylate, magnesium salicylate, salicyl salicylate, salicylamide, aspirin, arylalkoinic acids, diclofenac, aceclofenac, acemethacin, alclofenac, bromfenac, etodolac, indomethacin, nabumetone, oxametacin, proglumetacin, sulindac, tolmetin, 2-arylpropionic acids, profens, alminoprofen, benoxaprofen, carprofen, dexibuprofen, dexketoprofen, fenbufen, fenoprofen, flunoxaprofen, flurbiprofen, ibupromax, indoprofen, ketoprofen, ketorolac, loxoprofen, naproxen, oxaprozin, pirprofen, suprofen, tiaprofenic acid, ibuprofen, N-arylanthranilic acids, fenamic acids, mefenamic acid, flufenamic acid, meclofenamic acid, tolfenamic acid, phenylbutazone, ampyrone, azapropazone, clofezone, kebuzone, metamizole, mofebutazone, oxyphenbutazone, phenazone, sulfinpyrazone, oxicams, piroxicam, droxicam, lornoxicam, meloxicam, tenoxicam, celecoxib, etoricoxib, lumiracoxib, parecoxib, rofecoxib, valdecoxib, naproxen, or the like. Steroid include, by way of non-limiting example, corticosteroids, hydrocortisone, cortisone acetate, prednisone, prednisolone, methylprednisolone, dexamethasone, betamethasone, triamcinolone, beclometasone, fludrocortisone acetate, deoxycorticosterone acetate, aldosterone, or the like.

In specific embodiments, the additional therapeutic agent is an immunosuppressant. Immunosuppressants include, by way of non-limiting example, tacrolimus, cyclosporin, rapamicin, methotrexate, cyclophosphamide, azathioprine, mercaptopurine, mycophenolate, and FTY720.

In some embodiments, one or more of the anti-cancer agents are proapoptotic agents. Examples of anti-cancer agents include, by way of non-limiting example: gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, or PD184352, Taxol™, also referred to as “paclitaxel”, which is a well-known anti-cancer drug which acts by enhancing and stabilizing microtubule formation, and analogs of Taxol™, such as Taxotere™. Compounds that have the basic taxane skeleton as a common structure feature, have also been shown to have the ability to arrest cells in the G2-M phases due to stabilized microtubules and may be useful for treating cancer in combination with the compounds described herein.

Further examples of anti-cancer agents include inhibitors of mitogen-activated protein kinase signaling, e.g., U0126, PD98059, PD184352, PD0325901, ARRY-142886, SB239063, SP600125, BAY 43-9006, wortmannin, or LY294002; Syk inhibitors; mTOR inhibitors; and antibodies (e.g., rituxan).

Other anti-cancer agents include Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or r1L2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinzolidine sulfate; vinzolidine sulfate; vorozole; zeniplatin; zinostatin; zorubicin hydrochloride.

Other anti-cancer agents include: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin, nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Yet other anticancer agents that include alkylating agents, antimetabolites, natural products, or hormones, e.g., nitrogen mustards (e.g., mechloroethamine, cyclophosphamide, chlorambucil, etc.), alkyl sulfonates (e.g., busulfan), nitrosoureas (e.g., carmustine, lomusitne, etc.), or triazenes (decarbazine, etc.). Examples of antimetabolites include but are not limited to folic acid analog (e.g., methotrexate), or pyrimidine analogs (e.g., Cytarabine), purine analogs (e.g., mercaptopurine, thioguanine, pentostatin).

Examples of natural products include but are not limited to vinca alkaloids (e.g., vinblastin, vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha).

Examples of alkylating agents include, but are not limited to, nitrogen mustards (e.g., mechloroethamine, cyclophosphamide, chlorambucil, meiphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites include, but are not limited to folic acid analog (e.g., methotrexate), or pyrimidine analogs (e.g., fluorouracil, floxouridine, Cytarabine), purine analogs (e.g., mercaptopurine, thioguanine, pentostatin.

Examples of hormones and antagonists include, but are not limited to, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents that are optionally used in the methods and compositions described herein for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

In some embodiments, provided herein is a method of treating lymphoma comprising administering a therapeutically effective amount of a compound described herein in combination with an antibody to CD20 and/or a CHOP (cyclophosphamide, doxoruhicin, vincristine, and prednisone) therapy. In certain embodiments, provided herein is a method of treating leukemia comprising administering a therapeutically effective amount of a compound described herein in combination with ATRA, methotrexate, cyclophosphamide and the like.

### Pharmaceutical Compositions

In certain embodiments, pharmaceutical compositions are formulated in a conventional manner using one or more physiologically acceptable carriers including, e.g., excipients and auxiliaries which facilitate processing of the active compounds into preparations which are suitable for pharmaceutical use. In certain embodiments, proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

A pharmaceutical composition, as used herein, refers to a mixture of a compound described herein, such as, for example, a compound of any of Formulas I-V, with other chemical components, such as carriers, stabilizers, diluents, dispersing agents, suspending agents, thickening agents, and/or excipients. In certain instances, the pharmaceutical composition facilitates administration of the compound to an individual or cell. In certain embodiments of practicing the methods of treatment or use provided herein, therapeutically effective amounts of compounds described herein are administered in a pharmaceutical composition to an individual having a disease, disorder, or condition to be treated. In specific embodiments, the individual is a human. As discussed herein, the compounds described herein are either utilized singly or in combination with one or more additional therapeutic agents.

In certain embodiments, the pharmaceutical formulations described herein are administered to an individual in any manner, including one or more of multiple administration routes, such as, by way of non-limiting example, oral, parenteral (e.g., intravenous, subcutaneous, intramuscular), intranasal, buccal, topical, rectal, or transdermal administration routes.

In certain embodiments, a pharmaceutical compositions described herein includes one or more compound described herein, e.g., a compound of any of Formulas I-V, as an active ingredient in free-acid or free-base form, or in a pharmaceutically acceptable salt form. In some embodiments, the compounds described herein are utilized as an N-oxide or in a crystalline or amorphous form (i.e., a polymorph). In some situations, a compound described herein exists as tautomers. All tautomers are included within the scope of the compounds presented herein. In certain embodiments, a compound described herein exists in an unsolvated or solvated form, wherein solvated forms comprise any pharmaceutically acceptable solvent, e.g., water, ethanol, and the like. The solvated forms of the compounds presented herein are also considered to be disclosed herein.

A “carrier” includes, in some embodiments, a pharmaceutically acceptable excipient and is selected on the basis of compatibility with compounds disclosed herein, such as, compounds of any of Formulas I-V, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Moreover, in certain embodiments, the pharmaceutical compositions described herein is formulated as a dosage form. As such, in some embodiments, provided herein is a dosage form comprising a compound described herein, e.g., a compound of any of Formulas I-V, suitable for administration to an individual. In certain embodiments, suitable dosage forms include, by way of non-limiting example, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

The pharmaceutical solid dosage forms described herein optionally include an additional therapeutic compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In some aspects, using standard coating procedures, such as those described in Remington's Pharmaceutical Sciences, 20th Edition (2000), a film coating is provided around the formulation of the compound of any of Formula I-V. In one embodiment, a compound described herein is in the form of a particle and some or all of the particles of the compound are coated. In certain embodiments, some or all of the particles of a compound described herein are microencapsulated. In some embodiment, the particles of the compound described herein are not microencapsulated and are uncoated.

### EXAMPLES

### Example 1

### Synthesis of (E)-N-phenyl-2-(4-(4-(pyrrolidin-1-yl)but-2-enoyl)phenylamino)thiazole-5-carboxamide

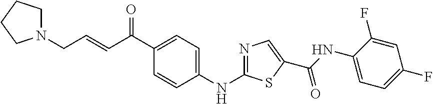

### Step 1: Synthesis of 2-(4-acetylphenylamino)-N-(2,4-difluorophenyl)-N-(4-methoxybenzyl)thiazole-5-carboxamide

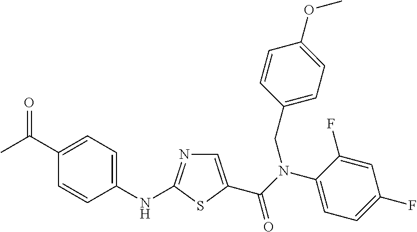

1-(4-aminophenyl)ethanone (1 g, 7.4 mmol) and NaH (60% by weight suspension in mineral oil) (0.35 g, 8.9 mmol) is stirred in 100 mL anhydrous THF for 10 min at 0° C. A solution of 2-bromo-N-(2,4-difluorophenyl)-N-(4-methoxybenzyl)thiazole-5-carboxamide (prepared according to procedure described in e.g. U.S. Pat. No. 6,596,746) in 25 mL THF is added to the reaction mixture and mixture is heated at 65° C. overnight. The reaction mixture is cooled to room temperature, quenched with 1N HCl and partitioned between EtOAc and water. The organic layer is separated and washed with brine, dried over Na₂SO₄, filtered, concentrated, and purified by column chromatography.

### Step 2: Synthesis of 2-(pyrrolidin-1-yl)acetaldehyde

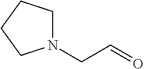

PCC (1 g, 4.6 mmol) is suspended in 100 mL dichloromethane. 2-(pyrrolidin-1-yl)ethanol (534 μL, 4.6 mmol) is added and the mixture is stirred at room temperature overnight. The reaction mixture is filtered through a florisil plug and the filtrate is concentrated.

### Step 3: Synthesis of (E)-N-(2,4-difluorophenyl)-N-(4-methoxybenzyl)-2-(4-(4-(pyrrolidin-1-yl)but-2-enoyl)phenylamino)thiazole-5-carboxamide

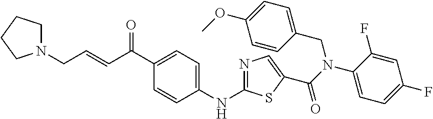

A solution of 2-(4-acetylphenylamino)-N-(2,4-difluorophenyl)-N-(4-methoxybenzyl)thiazole-5-carboxamide (0.4 g, 0.81 mmol) in 50 mL THF is cooled to 0° C. LDA (1M in THF, 820 μL, 1.6 mmol) is added dropwise and the mixture is stirred for 15 min at 0° C. A solution of 2-(pyrrolidin-1-yl)acetaldehyde (10 mg, 0.81 mmol) in 5 mL THF is added the reaction mixture and the mixture is allowed to warm to room temperature over 2 hours. The reaction is quenched with saturated NH₄Cl solution and partitioned between EtOAc and water. The organic layer is separated, washed with water, brine, dried over Na₂SO₄ and concentrated. The concentrate is suspended in a mixture of 1:1:1 tert-butanol:H₂O:H₂SO₄ and heated at 100° C. for 4 hours. The reaction is cooled to 0° C. and quenched with NaHCO₃. The mixture is extracted with EtOAc, the organic layer is separated, washed with water, brine, dried over Na₂SO₄, concentrated in vacuo and purified by column chromatography.

### Step 4: Synthesis of (E)-N-phenyl-2-(4-(4-(pyrrolidin-1-yl)but-2-enoyl)phenylamino)thiazole-5-carboxamide

(E)-N-(2,4-difluorophenyl)-N-(4-methoxybenzyl)-2-(4-(4-(pyrrolidin-1-yl)but-2-enoyl)phenylamino)thiazole-5-carboxamide (100 mg, 0.17 mmol) is dissolved in 9:1 ACN:H₂O. DDQ (39 mg, 0.17 mmol) is added to the reaction mixture and the mixture is stirred overnight. The reaction mixture is filtered through a celite plug and the filtrate is concentrated. The concentrate is purified by column chromatography.

The compounds set forth in FIG. 1 are synthesized according to a similar procedure as described in Example 1 using the appropriate starting materials and reagents.

### Example 2

### Synthesis of (E)-N-(4-chlorophenyl)-2-(1-(4-(pyrrolidin-1-yl)but-2-enoyl)piperidin-4-ylamino)thiazole-5-carboxamide

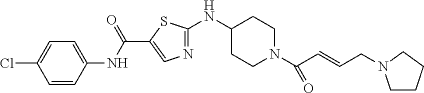

### Step 1: Synthesis of tert-butyl 4-(5-((4-chlorophenyl)(4-methoxybenzyl)carbamoyl)thiazol-2-ylamino)piperidine-1-carboxylate

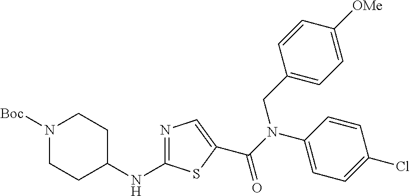

Tert-butyl 4-aminopiperidine-1-carboxylate (2 g, 9.9 mmol) and NaH (60% by weight suspension in mineral oil) (0.48 g, 11.9 mmol) is stirred in 100 mL anhydrous THF for 10 min at 0° C. A solution of 2-bromo-N-(4-chlorophenyl)-N-(4-methoxybenzyl)thiazole-5-carboxamide (prepared according to procedure described in e.g. U.S. Pat. No. 6,596,746) in 25 mL THF is added to the reaction mixture and mixture is heated at 65° C. overnight. The reaction mixture is cooled to room temperature, quenched with 1N HCl and partitioned between EtOAc and water. The organic layer is separated and washed with brine, dried over Na₂SO₄, filtered and concentrated and purified by column chromatography.

### Step 2: Synthesis of N-(4-chlorophenyl)-N-(4-methoxybenzyl)-2-(piperidin-4-ylamino)thiazole-5-carboxamide

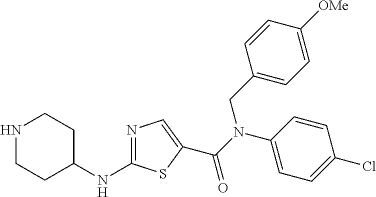

Tert-butyl 4-(5-((4-chlorophenyl)(4-methoxybenzyl)carbamoyl)thiazol-2-ylamino)piperidine-1-carboxylate (2 g, 3.6 mmol) is stirred in 50 mL TFA at room temperature for 3 h. The solution is concentrated in vacuo and then diluted with EtOAc. The EtOAc solution is washed with NaHCO₃ solution, water, brine, dried (MgSO₄), filtered and concentrated under reduced pressure.

### Step 3: Synthesis of (E)-4-(pyrrolidin-1-yl)but-2-enoyl chloride

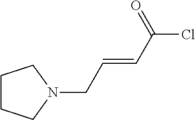

A solution of ethyl acetate (5 mL, 20 mmol) in 20 mL THF is cooled to 0° C. LDA (1M in THF, 20 mL, 20 mmol) is added dropwise and the mixture is stirred for 15 min at 0° C. A solution of 2-(pyrrolidin-1-yl)acetaldehyde (246 mg, 200 mmol) in 5 mL THF is added the reaction mixture and the mixture is allowed to warm to room temperature over 2 hours. The reaction is quenched with saturated NH₄Cl solution and partitioned between EtOAc and water. The organic layer is separated, washed with water, brine, dried over Na₂SO₄ and concentrated in vacuo. The concentrate is suspended in a mixture of 1:1:1 tert-butanol:H₂O:H₂SO₄ and heated at 100° C. for 4 hours. The reaction is cooled to 0° C. and diluted with water. The mixture is extracted with EtOAc, the organic layer is separated, washed with water, brine, dried over Na₂SO₄ and concentrated under reduced pressure. The concentrate (0.5 g, 3.2 mmol) is suspended in 20 mL dichloromethane. Oxalyl chloride (546 μL, 6.4 mmol) is added followed by two drops of DMF. The reaction is stirred at room temperature for 1.5 hr. The solvents are removed in vacuo.

### Step 4: Synthesis of (E)-N-(4-chlorophenyl)-N-(4-methoxybenzyl)-2-(1-(4-(pyrrolidin-1-yl)but-2-enoyl)piperidin-4-ylamino)thiazole-5-carboxamide

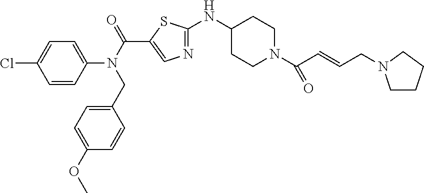

A solution of N-(4-chlorophenyl)-N-(4-methoxybenzyl)-2-(piperidin-4-ylamino)thiazole-5-carboxamide (1.2 g, 2.7 mmol) in 50 mL CH₂Cl₂ is added to a solution of solution of (E)-4-(pyrrolidin-1-yl)but-2-enoyl chloride (500 mg, 2.7 mmol) in 5 mL CH₂Cl₂. Triethyl amine (40 μL, 2.7 mmol) is added and the reaction mixture is stirred for 2 hr at room temperature. The reaction is partioned between CH₂Cl₂ and water. The organic layer is dried (MgSO₄), filtered, concentrated and purified by column chromatography.

### Step 5: Synthesis of (E)-N-(4-chlorophenyl)-2-(1-(3-(pyrrolidin-1-yl)acryloyl)piperidin-4-ylamino)thiazole-5-carboxamide

(E)-N-(4-chlorophenyl)-N-(4-methoxybenzyl)-2-(4-(4-(pyrrolidin-1-yl)but-2-enoyl)phenylamino)thiazole-5-carboxamide (100 mg, 0.17 mmol) is dissolved in 9:1 ACN:H₂O. DDQ (39 mg, 0.17 mmol) is added to the reaction mixture and the mixture is stirred overnight. The reaction mixture is filtered through a celite plug, the filtrate is concentrated and purified by column chromatography.

The compounds of FIG. 2 are synthesized according to a similar procedure as described in Example 2 using the appropriate starting materials and reagents.

### Example 3

### Synthesis of (E)-N-(4-chlorophenyl)-2-(4-(2-(phenylsulfonyl)vinyl)phenylamino)thiazole-5-carboxamide

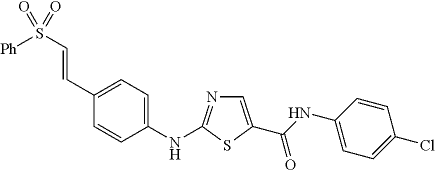

### Step 1: Synthesis of N-(4-chlorophenyl)-2-(4-formylphenylamino)-N-(4-methoxybenzyl)thiazole-5-carboxamide

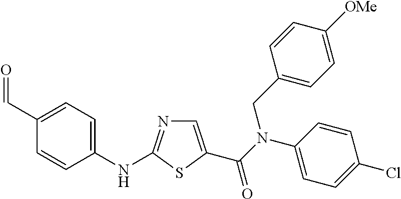

4-((tert-butyldimethylsilyloxy)methyl)aniline (2 g, 8.4 mmol) and NaH (60% by weight suspension in mineral oil) (0.35 g, 8.9 mmol) is stirred in 100 mL anhydrous THF for 10 min at 0° C. A solution of 2-bromo-N-(4-chlorophenyl)-N-(4-methoxybenzyl)thiazole-5-carboxamide (prepared according to procedure described in e.g. U.S. Pat. No. 6,596,746) in 25 mL THF is added to the reaction mixture and mixture is heated at 65° C. overnight. The reaction mixture is cooled to room temperature, quenched with 1N HCl and partitioned between EtOAc and water. The organic layer is separated and washed with brine, dried over Na₂SO₄, filtered and concentrated. The concentrate (1.5 g, 2.7 mmol) is dissolved in 50 mL THF. 2.7 mL of 1M TBAF in THF is added. The reaction mixture is stirred at room temp for 1 hr and partitioned between EtOAc and water. The organic layer is separated, dried (MgSO₄) and concentrated; the concentrate is dissolved in CH₂Cl₂. PCC (543 mg, 2.7 mmol) is added and the mixture is stirred overnight. The reaction mixture is filtered through a florisil pad and the filtrate is concentrated; the concentrate is triturated with ether.

### Step 2: Synthesis of (E)-N-(4-chlorophenyl)-N-(4-methoxybenzyl)-2-(4-(2-(phenylsulfonyl)vinyl)phenylamino)thiazole-5-carboxamide

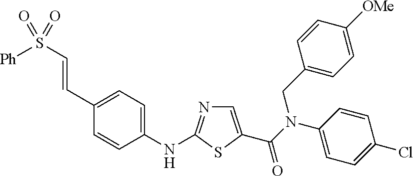

Diethyl(methylsulfonylmethyl)phosphonate (MSMP) is prepared according to the procedure described in U.S. Pat. No. 6,287,840. To a solution of MSMP (1.5 g, 6.51 mmol) is added sodium hydride (60% in mineral oil) (0.26 g, 13.4 mmol). The mixture is stirred for 15 minutes. N-(4-chlorophenyl)-2-(4-formylphenylamino)-N-(4-methoxybenzyl)thiazole-5-carboxamide (4.1 g, 6.7 mmol) is dissolved in 10 mL THF and added to the reaction mixture and the mixture was stirred for 1 hour. The reaction is quenched with 1N HCl and the mixture partitioned between EtOAc and water. The organic layer is separated, washed with water, brine, dried (MgSO₄) and filtered. The filtrate is concentrated and purified by recrystallization.

### Step 3: Synthesis of (E)-N-(4-chlorophenyl)-2-(4-(2-(phenylsulfonyl)vinyl)phenylamino)thiazole-5-carboxamide

(E)-N-(4-chlorophenyl)-N-(4-methoxybenzyl)-2-(4-(2-(phenylsulfonyl)vinyl)phenylamino)thiazole-5-carboxamide (250 mg, 0.41 mmol) is dissolved in 9:1 ACN:H₂O. DDQ (93 mg, 0.41 mmol) is added to the reaction mixture and the mixture is stirred overnight. The reaction mixture is filtered through a celite plug and the filtrate is concentrated and purified by column chromatography.

The compounds of FIG. 3 are synthesized according to a similar procedure as described in Example 3 using the appropriate starting materials and reagents.

### Example 4

### Synthesis of N-(4-chlorophenyl)-2-(4-(3-phenyloxiran-2-yl)phenylamino)thiazole-5-carboxamide

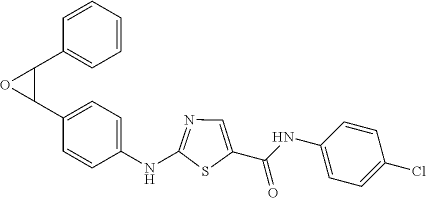

### Step 1: Synthesis of N-(4-chlorophenyl)-N-(4-methoxybenzyl)-2-(4-(3-phenyloxiran-2-yl)phenylamino)thiazole-5-carboxamide

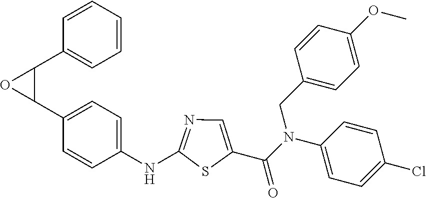

(Z)—N-(4-chlorophenyl)-N-(4-methoxybenzyl)-2-(4-styrylphenylamino)thiazole-5-carboxamide (prepared according to Example 3) (2 g, 3.6 mmol) is dissolved in 20 mL chloroform. m-chloroperbenzoic acid (670 mg, 3.9 mmol) is added to the solution and the reaction mixture is stirred for 4 hr at room temperature. The reaction is filtered through a celite plug and the filtrate is partitioned between water and chloroform. The organic layer is washed with water, brine, dried (Na₂SO₄), filtered, and the filtrate is concentrated and purified by recrystallization.

### Step 2: Synthesis of N-(4-chlorophenyl)-2-(4-(3-phenyloxiran-2-yl)phenylamino)thiazole-5-carboxamide

N-(4-chlorophenyl)-N-(4-methoxybenzyl)-2-(4-(3-phenyloxiran-2-yl)phenylamino)thiazole-5-carboxamide (100 mg, 0.17 mmol) is dissolved in 9:1 ACN:H₂O. DDQ (39 mg, 0.17 mmol) is added to the reaction mixture and the mixture is stirred overnight. The reaction mixture is filtered through a celite plug and the filtrate is concentrated and purified by column chromatography.

### Example 5

### Graft Rejection/Graft Versus Host Assay

Larynges are transplanted from Lewis-Brown-Norway (RT11/n, F1) donors to Lewis (RT11) recipients. Recipients receive 7 days of treatment with a Compound 100 analog modified or substituted with an electrophile subject to nucleophilic substitution or nucleophilic addition with a cysteine residue (e.g., a compound of any of Formulas I-V) and mouse anti-rat alphabeta T-cell-receptor (TCR) monoclonal antibodies. Histology, mixed lymphocyte reaction (MLR), skin grafting, and flow cytometry assess functional tolerance, efficacy of immunodepletion, and donor-specific chimerism. At 100 days, the survival rate, and allograft tolerance of the mice is determined Skin grafting, MLR, and flow cytometry are examined to confirm that tolerance is neither donor-specific nor related to systemic immunocompromise.

### Example 6

### Graft Rejection/Graft Versus Host Assay

Larynges are transplanted from Lewis-Brown-Norway (RT11/n, F1) donors to Lewis (RT11) recipients. Recipients receive 7 days of treatment with compound 42 and mouse anti-rat alphabeta T-cell-receptor (TCR) monoclonal antibodies. Histology, mixed lymphocyte reaction (MLR), skin grafting, and flow cytometry assess functional tolerance, efficacy of immunodepletion, and donor-specific chimerism. At 100 days, the survival rate, and allograft tolerance of the mice is determined Skin grafting, MLR, and flow cytometry are examined to confirm that tolerance is neither donor-specific nor related to systemic immunocompromise.

### Example 7

### Graft Rejection/Graft Versus Host Assay

Larynges are transplanted from Lewis-Brown-Norway (RT11/n, F1) donors to Lewis (RT11) recipients. Recipients receive 7 days of treatment with compound 29 and mouse anti-rat alphabeta T-cell-receptor (TCR) monoclonal antibodies. Histology, mixed lymphocyte reaction (MLR), skin grafting, and flow cytometry assess functional tolerance, efficacy of immunodepletion, and donor-specific chimerism. At 100 days, the survival rate, and allograft tolerance of the mice is determined Skin grafting, MLR, and flow cytometry are examined to confirm that tolerance is neither donor-specific nor related to systemic immunocompromise.

### Example 8

### Treatment of Lymphoma

Human Clinical Trial of the Safety and/or Efficacy of a Compound 100 analog modified or substituted with an electrophile subject to nucleophilic substitution or nucleophilic addition with a cysteine residue (e.g., a compound of any of Formulas I-V) therapy

Objective: To determine the safety and pharmacokinetics of administered a Compound 100 analog modified or substituted with an electrophile subject to nucleophilic substitution or nucleophilic addition with a cysteine residue (e.g., a compound of any of Formulas I-V)

Study Design: This will be a Phase I, single-center, open-label, randomized dose escalation study followed by a Phase II study in cancer patients with disease that can be biopsied (e.g., lymphoma). Patients should not have had exposure to MS electrophilically modified Compound 100 analog prior to the study entry. Patients must not have received treatment for their cancer within 2 weeks of beginning the trial. Treatments include the use of chemotherapy, hematopoietic growth factors, and biologic therapy such as monoclonal antibodies. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive oral electrophilically modified Compound 100 analog daily for 5 consecutive days or 7 days a week. Doses of electrophilically modified Compound 100 analog may be held or modified for toxicity based on assessments as outlined below. Treatment repeats every 28 days in the absence of unacceptable toxicity. Cohorts of 3-6 patients receive escalating doses of electrophilically modified Compound 100 analog until the maximum tolerated dose (MTD) for electrophilically modified Compound 100 analog is determined The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive electrophilically modified Compound 100 analog as in phase I at the MTD determined in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria.

Blood Sampling Serial blood is drawn by direct vein puncture before and after administration of electrophilically modified Compound 100 analog Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response to electrophilically modified Compound 100 analog therapy: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon the cancer type and feasibility/availability, and the same imaging modality is utilized for similar cancer types as well as throughout each patient's study course. Response rates are determined using the RECIST criteria or other similar response criteria. (Therasse et al, J. Natl. Cancer Inst. 2000 Feb. 2; 92(3):205-16; http://ctep.cancer.gov/forms/TherasseRECISTJNCI.pdf). Patients also undergo cancer/tumor biopsy to assess changes in progenitor cancer cell phenotype and clonogenic growth by flow cytometry, Western blotting, and IHC, and for changes in cytogenetics by FISH. After completion of study treatment, patients are followed periodically for 4 weeks.

### Example 9

### Treatment of Lymphoma

Human Clinical Trial of the Safety and/or Efficacy of compound 42 therapy

Objective: To compare the safety and pharmacokinetics of administered compound 42

Study Design: This will be a Phase I, single-center, open-label, randomized dose escalation study followed by a Phase II study in cancer patients with disease that can be biopsied (e.g., lymphoma). Patients should not have had exposure to MS compound 42 prior to the study entry. Patients must not have received treatment for their cancer within 2 weeks of beginning the trial. Treatments include the use of chemotherapy, hematopoietic growth factors, and biologic therapy such as monoclonal antibodies. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive oral compound 42 daily for 5 consecutive days or 7 days a week. Doses of compound 42 may be held or modified for toxicity based on assessments as outlined below. Treatment repeats every 28 days in the absence of unacceptable toxicity. Cohorts of 3-6 patients receive escalating doses of compound 42 until the maximum tolerated dose (MTD) for compound 42 is determined. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive compound 42 as in phase I at the MTD determined in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria.

Blood Sampling Serial blood is drawn by direct vein puncture before and after administration of compound 42 Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response to compound 42 therapy: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon the cancer type and feasibility/availability, and the same imaging modality is utilized for similar cancer types as well as throughout each patient's study course. Response rates are determined using the RECIST criteria or other similar response criteria. (Therasse et al, J. Natl. Cancer Inst. 2000 Feb. 2; 92(3):205-16; http://ctep.cancer.gov/forms/TherasseRECISTJNCI.pdf). Patients also undergo cancer/tumor biopsy to assess changes in progenitor cancer cell phenotype and clonogenic growth by flow cytometry, Western blotting, and IHC, and for changes in cytogenetics by FISH. After completion of study treatment, patients are followed periodically for 4 weeks.

### Example 10

### Treatment of Lymphoma

Human Clinical Trial of the Safety and/or Efficacy of compound 29 therapy

Objective: To compare the safety and pharmacokinetics of administered compound 29

Study Design: This will be a Phase I, single-center, open-label, randomized dose escalation study followed by a Phase II study in cancer patients with disease that can be biopsied (e.g., lymphoma). Patients should not have had exposure to MS compound 29 prior to the study entry. Patients must not have received treatment for their cancer within 2 weeks of beginning the trial. Treatments include the use of chemotherapy, hematopoietic growth factors, and biologic therapy such as monoclonal antibodies. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive oral compound 29 daily for 5 consecutive days or 7 days a week. Doses of compound 29 may be held or modified for toxicity based on assessments as outlined below. Treatment repeats every 28 days in the absence of unacceptable toxicity. Cohorts of 3-6 patients receive escalating doses of compound 29 until the maximum tolerated dose (MTD) for compound 29 is determined. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive compound 29 as in phase I at the MTD determined in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria.

Blood Sampling Serial blood is drawn by direct vein puncture before and after administration of compound 29 Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response to compound 29 therapy: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon the cancer type and feasibility/availability, and the same imaging modality is utilized for similar cancer types as well as throughout each patient's study course. Response rates are determined using the RECIST criteria or other similar response criteria. (Therasse et al, J. Natl. Cancer Inst. 2000 Feb. 2; 92(3):205-16; http://ctep.cancer.gov/forms/TherasseRECISTJNCI.pdf). Patients also undergo cancer/tumor biopsy to assess changes in progenitor cancer cell phenotype and clonogenic growth by flow cytometry, Western blotting, and IHC, and for changes in cytogenetics by FISH. After completion of study treatment, patients are followed periodically for 4 weeks.

### Example 11

### Treatment of Leukemia

Human Clinical Trial of the Safety and/or Efficacy of a Compound 100 analog modified or substituted with an electrophile subject to nucleophilic substitution or nucleophilic addition with a cysteine residue (e.g., a compound of any of Formulas I-V) therapy

Objective: To determine the safety and pharmacokinetics of administered electrophilically modified Compound 100 analog

Study Design: This will be a Phase I, single-center, open-label, randomized dose escalation study followed by a Phase II study in leukemia patients. Patients should not have had exposure to electrophilically modified Compound 100 analog prior to the study entry. Patients must not have received treatment for their cancer within 2 weeks of beginning the trial. Treatments include the use of chemotherapy, hematopoietic growth factors, and biologic therapy such as monoclonal antibodies. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive oral electrophilically modified Compound 100 analog daily for 5 consecutive days or 7 days a week. Doses of electrophilically modified Compound 100 analog may be held or modified for toxicity based on assessments as outlined below. Treatment repeats every 28 days in the absence of unacceptable toxicity. Cohorts of 3-6 patients receive escalating doses of electrophilically modified Compound 100 analog until the maximum tolerated dose (MTD) for the electrophilically modified Compound 100 analog is determined. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive electrophilically modified Compound 100 analog as in phase I at the MTD determined in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria.

Blood Sampling Serial blood is drawn by direct vein puncture before and after administration of electrophilically modified Compound 100 analog Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response to electrophilically modified Compound 100 analog therapy: Patient response is assessed with complete blood counts and differential (CBC) and/or bone marrow aspiration/biopsy and is performed prior to beginning the study and at the end of the first cycle, with additional bone marrow aspiration/biopsy performed every four weeks or at the end of subsequent cycles. Patients also undergo biopsy to assess changes in progenitor cancer cell phenotype and clonogenic growth by flow cytometry, and for changes in cytogenetics by FISH as a means to measure tumor burden. After completion of study treatment, patients are followed periodically for 4 weeks.

### Example 12

### Treatment of Leukemia

Human Clinical Trial of the Safety and/or Efficacy of compound 42 therapy

Objective: To determine the safety and pharmacokinetics of administered compound 42

Study Design: This will be a Phase I, single-center, open-label, randomized dose escalation study followed by a Phase II study in leukemia patients. Patients should not have had exposure to compound 42 prior to the study entry. Patients must not have received treatment for their cancer within 2 weeks of beginning the trial. Treatments include the use of chemotherapy, hematopoietic growth factors, and biologic therapy such as monoclonal antibodies. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive oral compound 42 daily for 5 consecutive days or 7 days a week. Doses of compound 42 may be held or modified for toxicity based on assessments as outlined below. Treatment repeats every 28 days in the absence of unacceptable toxicity. Cohorts of 3-6 patients receive escalating doses of compound 42 until the maximum tolerated dose (MTD) for the compound 42 is determined. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive compound 42 as in phase I at the MTD determined in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria.

Blood Sampling Serial blood is drawn by direct vein puncture before and after administration of compound 42 Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response to compound 42 therapy: Patient response is assessed with complete blood counts (CBC) and differential and/or bone marrow aspiration/biopsy and is performed prior to beginning the study and at the end of the first cycle, with additional bone marrow aspiration/biopsy performed every four weeks or at the end of subsequent cycles. Patients also undergo biopsy to assess changes in progenitor cancer cell phenotype and clonogenic growth by flow cytometry, and for changes in cytogenetics by FISH as a means to measure tumor burden. After completion of study treatment, patients are followed periodically for 4 weeks.

### Example 13

### Treatment of Leukemia

Human Clinical Trial of the Safety and/or Efficacy compound 29 therapy

Objective: To determine the safety and pharmacokinetics of administered compound 29

Study Design: This will be a Phase I, single-center, open-label, randomized dose escalation study followed by a Phase II study in leukemia patients. Patients should not have had exposure to compound 29 prior to the study entry. Patients must not have received treatment for their cancer within 2 weeks of beginning the trial. Treatments include the use of chemotherapy, hematopoietic growth factors, and biologic therapy such as monoclonal antibodies. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive oral compound 29 daily for 5 consecutive days or 7 days a week. Doses of compound 29 may be held or modified for toxicity based on assessments as outlined below. Treatment repeats every 28 days in the absence of unacceptable toxicity. Cohorts of 3-6 patients receive escalating doses of compound 29 until the maximum tolerated dose (MTD) for the compound 29 is determined. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive compound 29 as in phase I at the MTD determined in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria.

Blood Sampling Serial blood is drawn by direct vein puncture before and after administration of compound 29 Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response to compound 29 therapy: Patient response is assessed with complete blood counts and differential (CBC) and/or bone marrow aspiration/biopsy and is performed prior to beginning the study and at the end of the first cycle, with additional bone marrow aspiration/biopsy performed every four weeks or at the end of subsequent cycles. Patients also undergo biopsy to assess changes in progenitor cancer cell phenotype and clonogenic growth by flow cytometry, and for changes in cytogenetics by FISH as a means to measure tumor burden. After completion of study treatment, patients are followed periodically for 4 weeks.

### Example 14

### Drug Screening Assay

Protein kinase activity results in the incorporation of radio labeled Compound 100 (e.g., tritiated Compound 100) into a peptide or protein substrate. The measurement of the amount of radioactivity incorporated into a substrate as a function of time, kinase (e.g., BTK, BMX, TEC, TXK, ITK, EGFR, ErbB2, ErbB4, JAK3, and/or BLK) concentration, and radiolabel Compound 100 concentration allows radio labeled Compound 100 activity to be quantified (and utilized as a standard or control). The activity is expressed as a ‘unit’, where 1 unit corresponds to the amount of protein kinase that catalyzes the incorporation of 1 nanomole of phosphate into the standard substrate in 1 minute. Specific activity is defined as units of activity per milligram protein. Up to 40 samples can be assayed manually at one time, and the assay takes one person less than 1 hour to complete. (See, e.g., Nature Protocols 1, 968-971 (2006).)

In one instance, radio labeled Compound 100 is contacted with a select protein kinase (e.g., BTK, BMX, TEC, TXK, ITK, EGFR, ErbB2, ErbB4, JAK3, and/or BLK) in combination with a candidate compound (e.g., an electrophilically enhanced Compound 100 compound, such as one set for in any of Formulas I-V). The measurement of the amount of radioactivity incorporated into a substrate as a function of time, kinase (e.g., BTK, BMX, TEC, TXK, ITK, EGFR, ErbB2, ErbB4, JAK3, and/or BLK) concentration, radio labeled Compound 100 concentration, and candidate compound concentration allows the activity (e.g., with respect to Compound 100) of the candidate compound to be quantified. In particular, over time, in some instances, a candidate compound that irreversibly binds the kinase, the concentration of the radio labeled Compound 100 bound to the kinase may decrease over time. In other instances, a candidate compound that has significantly greater activity than Compound 100 may provide an assay wherein the radio labeled Compound 100 does not show a significant amount of binding to the kinase at any time.

Alternatively, radio labeled candidate compounds (e.g., tritiated) are optionally utilized and their activities are directly measured.

### Example 15

### Luminescence-Based Kinase Assay

This assay makes use of an ATP depletion assay (Kinase-Glo®, Promega Corporation, Madison, Wis.) to quantitate kinase activity of a candidate compound (e.g., an electrophilically enhanced Compound 100 compound, such as one set for in any of Formulas I-V).

The following stock solutions are prepared: 10 mM solution of compound 100; 100 mM HEPES buffer, pH 7.5 (5 ml 1M stock+45 ml miliQH₂O); 10 mM ATP (5.51 mg/ml in dH₂O) (diluted 50 μl into total of 10 ml miliQH₂O daily=50 μM ATP working stock); 1% BSA (1 g BSA in 100 ml 0.1 M HEPES, pH 7.5), 100 mM MgCl₂; 200 μM Staurosporine, 2xKinase-Glo® reagent.

Standard Assay Setup for 384-well format (20 μl kinase reaction, 40 μl detection reaction): 10 mM MgCl₂, 100 μM Compound 100; 0.1% BSA; 1 μl candidate compound (in DMSO); 0.4 μg/ml kinase domain; 10 μM ATP; 100 mM HEPES buffer. Positive controls contain DMSO with no test compound. Negative controls contain 10 μM staurosporine. The kinase reactions are initiated at time t=0 by the addition of ATP. Kinase reactions are incubated at 21° C. for 30 min, then 20 μl of Kinase-Glo™ reagent is added to each well to quench the kinase reaction and initiate the luminescence reaction. After a 20 min incubation at 21° C., the luminescence is detected in a plate-reading luminometer. The luminescent signal from ATP remaining in solution following the kinase reaction is detected in a plate-reading luminometer. The luminescent signal is inversely correlated with the amount of kinase activity. Activity is optionally compared to the activity of compound 100.

### Example 16

### Computational Assays

Computational assays are used to identify compounds with a strong interaction (e.g., strongest interaction and/or best fit). The test compounds are screened through protein crystallographic screening, as disclosed in, for example Antonysamy, et al., PCT Publication No. WO03087816A1, which is incorporated herein by reference.

Docking programs such as, for example, DOCK, or GOLD, are used to identify compounds that bind to the active site and/or other binding pockets. Compounds are screened against more than one binding pocket of the protein structure, or more than one set of coordinates for the same protein, taking into account different molecular dynamic conformations of the protein. Consensus scoring is used to identify the compounds that are the best fit for the protein (Charifson, P. S. et al., J. Med. Chem. 42: 5100-9 (1999)). Data obtained from more than one protein molecule structure is scored according to the methods described in Klingler et al., U.S. Utility Application, filed May 3, 2002, entitled “Computer Systems and Methods for Virtual Screening of Compounds.”

Electrophilic or other binding groups in test compounds are computationally evaluated by means of a series of steps in which chemical groups or fragments are screened and selected for their ability to associate with the individual binding pockets, residues or other areas of kinases. Selected fragments or chemical groups are positioned in a variety of orientations, or docked, within binding pockets of kinases (Blaney, J. M. and Dixon, J. S., Perspectives in Drug Discovery and Design, 1:301, 1993). Manual docking is accomplished using any suitable software, such as Insight II (Accelrys, San Diego, Calif.) MOE (Chemical Computing Group, Inc., Montreal, Quebec, Canada); and SYBYL (Tripos, Inc., St. Louis, Mo., 1992), followed by energy minimization and/or molecular dynamics with standard molecular mechanics force fields, such as CHARMM (Brooks, et al., J. Comp. Chem. 4:187-217, 1983), AMBER (Weiner, et al., J. Am. Chem. Soc. 106: 765-84, 1984) and C.sup.2 MMFF (Merck Molecular Force Field; Accelrys, San Diego, Calif.). Other automated docking programs such as DOCK (Kuntz et al., J. Mol. Biol., 161:269-88, 1982; DOCK is available from University of California, San Francisco, Calif.); AUTODOCK (Goodsell & Olsen, Proteins: Structure, Function, and Genetics 8:195-202, 1990; AUTODOCK is available from Scripps Research Institute, La Jolla, Calif.); GOLD (Cambridge Crystallographic Data Centre (CCDC); Jones et al., J. Mol. Biol. 245:43-53, 1995); and FLEXX (Tripos, St. Louis, Mo.; Rarey, M., et al., J. Mol. Biol. 261:470-89, 1996) are used to screen compounds.

Evaluation of compound deformation energy and electrostatic interaction is accomplished using any suitable program such as Gaussian 94, revision C (Frisch, Gaussian, Inc., Pittsburgh, Pa. © 1995); AMBER, version 7. (Kollman, University of California at San Francisco, © 2002); QUANTA/CHARMM (Accelrys, Inc., San Diego, Calif., © 1995); Insight II/Discover (Accelrys, Inc., San Diego, Calif., © 1995); DelPhi (Accelrys, Inc., San Diego, Calif., © 1995); and AMSOL (University of Minnesota) (Quantum Chemistry Program Exchange, Indiana University).

1. A compound of Formula I: 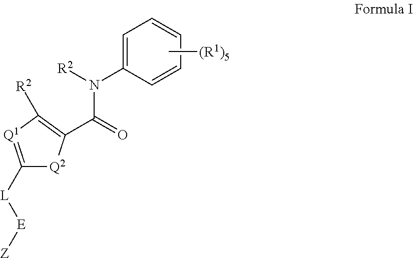
wherein:
each R¹ is independently H, alkyl, halo, hydroxy, alkoxy, cyano, nitro, C(═X)YR², or YC(═X)R²;
each X is independently S or O;
each Y is independently S or O;
each R² is independently H or alkyl;
L is Aₙ, wherein
each A is independently NR¹, substituted or unsubstituted aryl, substituted or unsubstituted heteroaryl, substituted or unsubstituted cycloalkyl, or substituted or unsubstituted heterocycloalkyl; wherein each m is independently 0-2;
n is 0-5;
Q¹ is N or CR²;
Q² is NR², S, or O;
E —(CR¹¹R¹²)ᵣ—(CR⁵═CR⁵)_{q}—(CR¹¹R¹²)ᵣ—, —(CR⁶R⁷)—X², —NR⁸(C═O)O—; —O(C═O)NR⁸—; —CR⁸R¹³(C═O)—; or —CR⁸R¹³(C═O)—,
R¹¹ and R¹² are independently H, CN, NO₂, substituted or unsubstituted alkyl, substituted or unsubstituted aryl, or substituted or unsubstituted heteroaryl, or taken together are ═S, ═N—OR⁸, or ═O; wherein each R⁸ is independently substituted or unsubstituted alkyl, substituted or unsubstituted aryl, substituted or unsubstituted cycloalkyl, substituted or unsubstituted heteroaryl, substituted or unsubstituted heterocycloalkyl, or substituted or unsubstituted heteroalkyl;
each R⁵ is independently H, halo, hydroxy, alkoxy, cyano, nitro, S(O)₁₋₂R⁸, —C(═X)YR⁸,
—YC(═X)R⁸, substituted or unsubstituted alkyl, substituted or unsubstituted aryl, substituted or unsubstituted cycloalkyl, substituted or unsubstituted heteroaryl, substituted or unsubstituted heterocycloalkyl, substituted or unsubstituted heteroalkyl, or two R⁵ are taken together to form a bond;
each r is independently 0-2;
q is 0-2;
R⁶ and R⁷ are independently H, halo, hydroxy, alkoxy, cyano, nitro, —C(═X)YR⁸,
—YC(═X)R⁸, substituted or unsubstituted alkyl, substituted or unsubstituted aryl, substituted or unsubstituted cycloalkyl, substituted or unsubstituted heteroaryl, substituted or unsubstituted heterocycloalkyl, substituted or unsubstituted heteroalkyl, or is a bond to Z; or R⁶ and R⁷ taken together are ═O or ═S;
X² is halo, OR⁹, NR⁹ᵥ, N₃, SR⁹, or SCN; wherein R⁹ is —(S(O)ₜ)ᵤ—R¹⁰; wherein each R¹⁰ is independently H, substituted or unsubstituted alkyl, substituted or unsubstituted aryl, substituted or unsubstituted cycloalkyl, substituted or unsubstituted heteroaryl, substituted or unsubstituted heterocycloalkyl, or substituted or unsubstituted heteroalkyl; or X² and R⁷ when taken together with the carbon to which they are bound form an oxirane or oxetane; wherein t is 1-2, wherein u is 0-1, wherein v is 2-3;
R¹³ is halo;
Z is —(Z¹)ₚ—Z² or is absent,
Z¹ is substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl
Z² is H, NR³₂, S(O)ₘR³, OR³, —C(═X)YR³, —Y(C═X)R³, substituted or unsubstituted cycloalkyl, or substituted or unsubstituted heterocycloalkyl;
each R³ is independently H, halo, hydroxy, alkoxy, cyano, nitro, —C(═X)YR⁴, —YC(═X)R⁴, substituted or unsubstituted alkyl, substituted or unsubstituted aryl, substituted or unsubstituted cycloalkyl, substituted or unsubstituted heteroaryl, substituted or unsubstituted heterocycloalkyl, or substituted or unsubstituted heteroalkyl, wherein R⁴ is substituted or unsubstituted alkyl, substituted or unsubstituted aryl, substituted or unsubstituted cycloalkyl, substituted or unsubstituted heteroaryl, substituted or unsubstituted heterocycloalkyl, or substituted or unsubstituted heteroalkyl;
p is 0-4;
or a pharmaceutically acceptable salt thereof.

2. (canceled)

3. (canceled)

4. The compound of claim 1, wherein each R¹ is independently H, halo or alkyl.

5. The compound of claim 1, wherein Q¹ is N.

6. The compound of claim 1, wherein n is 1-2.

7. The compound of claim 1, wherein E is —(C═O)—(CH═CH)—, —(CH═CH)—(C═O)—, —C(CN)═CH—, —CH═C(CN)—, —C(NO₂)═CH—, or —CH═C(NO₂)—.

8. The compound of claim 1, wherein n is 2, and wherein one A is tetrahydroquinolinyl, quinolinyl, tetrahydroisoquinolinyl, isoquinolinyl, pyridinyl, piperazinyl, or morpholino.

9. The compound of claim 1, wherein Z² is a substituted or unsubstituted piperazinyl, or a substituted or unsubstituted morpholino.

10. The compound of claim 1 having the Formula II: 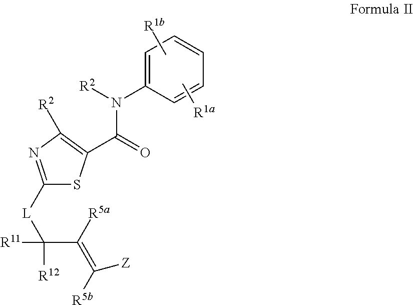
wherein
R^{1a} is H, halo, or lower alkyl;
R^{2a} is H, halo, or lower alkyl;
R¹¹ is H;
R¹² is H; or R¹¹ and R¹² taken together are ═O;
R^{5a} is H, lower alkyl, CN, NO₂, or SO₂R⁸; and
R^{5b} is H, CN, NO₂, or SO₂R⁸.

11. The compound of claim 10, wherein R^{1a} is CH₃ and R^{1b} is Cl.

12. The compound of claim 10, wherein R¹¹ and R¹² taken together are ═O, and wherein R^{5a} and R^{5b} are H.

13. A pharmaceutical composition comprising a therapeutically effective amount of a compound of Formula I: 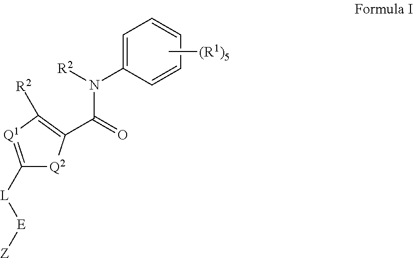
wherein:
each R¹ is independently H, alkyl, halo, hydroxy, alkoxy, cyano, nitro, C(═X)YR², or YC(═X)R²;
each X is independently S or O;
each Y is independently S or O;
each R² is independently H or alkyl;
L is Aₙ, wherein
each A is independently NR¹, S(O)ₘ, O, C(═X)Y, Y(C═X), substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl; wherein each m is independently 0-2;
n is 0-5;
Q¹ is N or CR²;
Q² is NR², S, or O;
E is an electrophile;
Z is —(Z¹)ₚ—Z² or is absent,
Z¹ is NR³, O, C(═X)Y, Y(C═X), substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl
Z² is H, NR³₂, S(O)ₘR³, OR³, —C(═X)YR³, —Y(C═X)R³, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
each R³ is independently H, halo, hydroxy, alkoxy, cyano, nitro, —C(═X)YR⁴, —YC(═X)R⁴, substituted or unsubstituted alkyl, substituted or unsubstituted aryl, substituted or unsubstituted cycloalkyl, substituted or unsubstituted heteroaryl, substituted or unsubstituted heterocycloalkyl, or substituted or unsubstituted heteroalkyl, wherein R⁴ is substituted or unsubstituted alkyl, substituted or unsubstituted aryl, substituted or unsubstituted cycloalkyl, substituted or unsubstituted heteroaryl, substituted or unsubstituted heterocycloalkyl, or substituted or unsubstituted heteroalkyl;
p is 0-4;
or a pharmaceutically acceptable salt thereof;
and a pharmaceutically acceptable carrier.

14. A method of treating a disorder mediated by a cysteine containing kinase comprising administering to an individual in need thereof a therapeutically effective amount of a compound of Formula I: 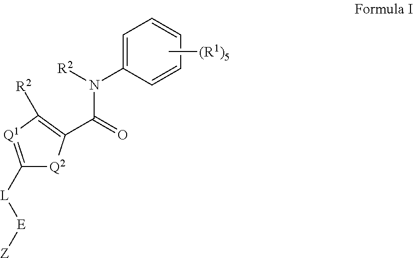
wherein:
each R¹ is independently H, alkyl, halo, hydroxy, alkoxy, cyano, nitro, C(═X)YR², or YC(═X)R²;
each X is independently S or O;
each Y is independently S or O;
each R² is independently H or alkyl;
L is Aₙ, wherein
each A is independently NR¹, S(O)ₘ, O, C(═X)Y, Y(C═X), substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl; wherein each m is independently 0-2;
n is 0-5;
Q¹ is N or CR²;
Q² is NR², S, or O;
E is an electrophile;
Z is —(Z¹)ₚ—Z² or is absent,
Z¹ is NR³, O, C(═X)Y, Y(C═X), substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl
Z² is H, NR³₂, S(O)ₘR³, OR³, —C(═X)YR³, —Y(C═X)R³, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
each R³ is independently H, halo, hydroxy, alkoxy, cyano, nitro, —C(═X)YR⁴, —YC(═X)R⁴, substituted or unsubstituted alkyl, substituted or unsubstituted aryl, substituted or unsubstituted cycloalkyl, substituted or unsubstituted heteroaryl, substituted or unsubstituted heterocycloalkyl, or substituted or unsubstituted heteroalkyl, wherein R⁴ is substituted or unsubstituted alkyl, substituted or unsubstituted aryl, substituted or unsubstituted cycloalkyl, substituted or unsubstituted heteroaryl, substituted or unsubstituted heterocycloalkyl, or substituted or unsubstituted heteroalkyl;
p is 0-4;
or a pharmaceutically acceptable salt thereof.

15. The method of claim 14, wherein the cysteine containing kinase comprises a cysteine in proximity to the ATP binding site of the kinase.

16. The method of claim 14, wherein the cysteine containing kinase is BTK, BMX, TEC, TXK, ITK, EGFR, ErbB2, ErbB4, JAK3, or BLK.

17. The method of claim 14, wherein the disorder is cancer, an inflammatory disorder, or an autoimmune disorder mediated by the cysteine containing kinase.

18. A method of binding a cysteine containing kinase to a compound of Formula I comprising contacting the kinase with the compound of Formula I, wherein the compound of Formula I has the structure: 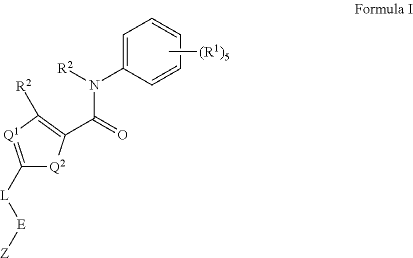
wherein:
each R¹ is independently H, alkyl, halo, hydroxy, alkoxy, cyano, nitro, C(═X)YR², or YC(═X)R²;
each X is independently S or O;
each Y is independently S or O;
each R² is independently H or alkyl;
L is Aₙ, wherein
each A is independently NR¹, S(O)ₘ, O, C(═X)Y, Y(C═X), substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl; wherein each m is independently 0-2;
n is 0-5;
Q¹ is N or CR²;
Q² is NR², S, or O;
E is an electrophile;
Z is —(Z¹)ₚ—Z² or is absent,
Z¹ is NR³, O, C(═X)Y, Y(C═X), substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl
Z² is H, NR³₂, S(O)ₘR³, OR³, —C(═X)YR³, —Y(C═X)R³, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
each R³ is independently H, halo, hydroxy, alkoxy, cyano, nitro, —C(═X)YR⁴, —YC(═X)R⁴, substituted or unsubstituted alkyl, substituted or unsubstituted aryl, substituted or unsubstituted cycloalkyl, substituted or unsubstituted heteroaryl, substituted or unsubstituted heterocycloalkyl, or substituted or unsubstituted heteroalkyl, wherein R⁴ is substituted or unsubstituted alkyl, substituted or unsubstituted aryl, substituted or unsubstituted cycloalkyl, substituted or unsubstituted heteroaryl, substituted or unsubstituted heterocycloalkyl, or substituted or unsubstituted heteroalkyl;
p is 0-4;
or a pharmaceutically acceptable salt thereof.

19. The method of claim 18, wherein the cysteine containing kinase is BTK, BMX, TEC, TXK, ITK, EGFR, ErbB2, ErbB4, JAK3, or BLK.

20. The method of claim 18, wherein the kinase is contacted with the compound of Formula I in vivo.
